(19) United States
(12) Patent Application Publication
(10) Pub. No.: US 2025/0331937 A1
(43) Pub. Date: Oct. 30, 2025

(54) System And Method For Aligning An End Effector To A Haptic Object

(71) Applicant: MAKO Surgical Corp., Weston, FL (US)

(72) Inventors: Mohamad Ahmadi, Waldkirch (DE); Matthias Wapler, Kirchzarten (DE); Lars Marius Schwalbe, Bollschweil (DE); Radu Iorgulescu, Boca Raton, FL (US); Shawn D. Stad, Lakeville, MA (US)

(73) Assignee: MAKO Surgical Corp., Weston, FL (US)

(21) Appl. No.: 19/188,452

(22) Filed: Apr. 24, 2025

Related U.S. Application Data
(60) Provisional application No. 63/638,713, filed on Apr. 25, 2024.
(60) Provisional application No. 63/669,329, filed on Jul. 10, 2024.

(51) Int. Cl. A61B 34/32; A61B 34/00; A61B 34/10; A61B 34/20; A61B 34/30; A61B 34/37

(52) U.S. Cl. CPC A61B 34/32; A61B 34/20; A61B 34/25; A61B 34/37; A61B 34/76; A61B 2034/107; A61B 2034/302

(57) ABSTRACT

A surgical system is provided. The surgical system includes a robotic arm, a navigation system, and one or more controllers. The robotic arm includes a plurality of links and joints and is configured to support an end effector. The navigation system is configured to track a pose of an anatomy of a patient. The one or more controllers are configured to associate a target trajectory with the anatomy of the patient, define a trajectory selection zone associated with the target trajectory, operate the robotic arm in a free mode, whereby the robotic arm is freely moveable, responsive to the end effector being within the trajectory selection zone in the free mode, automatically select the target trajectory associated with the trajectory selection zone, and operate the robotic arm in an automatic mode, whereby the robotic arm is automatically moved to align the end effector with the target trajectory.

### CROSS REFERENCE TO RELATED APPLICATIONS

The subject application claims priority to and all the benefits of U.S. Provisional Patent App. No. 63/638,713, filed Apr. 25, 2024, and claims priority to and all the benefits of U.S. Provisional Patent App. No. 63/669,329, filed Jul. 10, 2024, the entire contents of which are hereby incorporated by reference.

### BACKGROUND

Robotic systems for performing surgical procedures in a patient's anatomy are well known. For instance, robotic systems are currently utilized to place pedicle screws in a patient's anatomy.

When a patient requires surgery that involves placing pedicle screws, pre-operative imaging and/or intra-operative imaging is often employed to visualize the patient's anatomy that requires treatment. A surgeon then plans where to place the pedicle screws with respect to the images and/or with respect to a 3-D model created from the images. Planning includes determining a position and/or orientation (i.e., pose) of each pedicle screw with respect to the particular anatomy in which they are being placed, e.g., by identifying the desired pose in the images and/or the 3-D model. Once the plan is set, then the plan is transferred to the robotic system for execution.

Typically, the robotic system comprises a robotic manipulator that positions a tool based on a haptic object. The robotic system also comprises a navigation system to determine a location of the tool with respect to the patient's anatomy so that the robotic manipulator can place the tool based on the haptic object and according to the surgeon's plan.

However, there remain a need in the art for providing a more ergonomic and surgeon-friendly method of selecting a haptic object for positioning of the tool.

### SUMMARY

This Summary introduces a selection of concepts in a simplified form that are further described below in the Detailed Description below. This Summary is not intended to limit the scope of the claimed subject matter nor identify key features or essential features of the claimed subject matter.

In a first aspect, a surgical system is provided. The surgical system comprises: a robotic arm comprising a plurality of links and joints and being configured to support an end effector; a navigation system configured to track a pose of an anatomy of a patient; and one or more controllers configured to: associate a target trajectory with the anatomy of the patient; define a trajectory selection zone associated with the target trajectory; operate the robotic arm in a free mode, whereby the robotic arm is freely moveable; responsive to the end effector being within the trajectory selection zone in the free mode, automatically select the target trajectory associated with the trajectory selection zone; and operate the robotic arm in an automatic mode, whereby the robotic arm is automatically moved to align the end effector with the target trajectory.

In a second aspect, a surgical system is provided. The surgical system comprises: a navigation system comprising a localizer configured to track a pose of an anatomy; a robotic arm configured to support and move an end effector; an input device; and one or more controllers configured to: associate a haptic object to the anatomy, wherein a pose of the haptic object is dependent on the tracked pose of the anatomy; responsive to detecting a first input from the input device and responsive to the end effector being aligned with the haptic object, constrain the end effector to the haptic object; and responsive to detecting a second input from the input device and responsive to the end effector being aligned with the haptic object, constrain the end effector independent of the tracked pose of the anatomy.

In a third aspect, a surgical system is provided. A surgical system comprising: a robotic arm comprising a plurality of links and joints and being configured to support an end effector; a navigation system configured to track a pose of an anatomy of a patient; a display; and one or more controllers configured to: associate a first and second target trajectory with the anatomy of the patient; define a first trajectory selection zone associated with the first target trajectory and a second trajectory selection zone associated with the second target trajectory; and determine whether the end effector is closer to the first target trajectory or the second target trajectory; wherein the display is configured to indicate whether the end effector is closer to the first target trajectory or the second target trajectory.

In a fourth aspect, a surgical system is provided. The surgical system comprises: a robotic arm comprising a plurality of links and joints and being configured to support an end effector, wherein the end effector includes a guide tube configured to support an instrument temporarily affixed to the guide tube; a navigation system configured to track a pose of an anatomy of a patient; and one or more controllers configured to: associate a target trajectory with the anatomy of the patient; define a trajectory selection zone associated with the target trajectory; determine whether an instrument is temporarily affixed to the guide tube; and responsive to determining that an instrument is temporarily affixed to the guide tube and responsive to the guide tube being aligned with the target trajectory, operate the robotic arm in a haptic mode to constrain movement of the guide tube to the target trajectory and prevent movement of the guide tube away from the anatomy of the patient.

In a fifth aspect, a surgical system is provided. The surgical system comprises: a robotic arm comprising a plurality of links and joints and being configured to support an end effector; a navigation system configured to track a pose of an anatomy of a patient; and one or more controllers configured to: associate a haptic object with the anatomy of the patient; define a selection zone associated with the haptic object; operate the robotic arm in a free mode, whereby the robotic arm is freely moveable; responsive to the end effector being within the selection zone in the free mode, automatically select the haptic object associated with the selection zone; and operate the robotic arm in an automatic mode, whereby the robotic arm is automatically moved to align the end effector with the haptic object.

In a sixth aspect, a surgical system is provided. The surgical system comprises: a robotic arm comprising a plurality of links and joints and being configured to support an end effector; a navigation system configured to track a pose of an anatomy of a patient; and one or more controllers configured to: associate a virtual cutting plane with the anatomy of the patient; define a selection zone associated with the virtual cutting plane; operate the robotic arm in a free mode, whereby the robotic arm is freely moveable; responsive to the end effector being within the selection zone in the free mode, automatically select the virtual cutting plane associated with the selection zone; and operate the robotic arm in an automatic mode, whereby the robotic arm is automatically moved to align the end effector with the virtual cutting plane.

In a seventh aspect, a surgical system is provided. The surgical system comprises: a robotic arm comprising a plurality of links and joints and being configured to support an end effector; a navigation system configured to track a pose of an anatomy of a patient; and one or more controllers configured to: associate a target trajectory with the anatomy of the patient; define a trajectory selection zone associated with the target trajectory; operate the robotic arm in a free mode, whereby the robotic arm is freely moveable; responsive to the end effector being within the trajectory selection zone in the free mode, automatically select the target trajectory associated with the trajectory selection zone; implement haptic feedback to indicate that the target trajectory is selected; and operate the robotic arm in an automatic mode, whereby the robotic arm is automatically moved to align the end effector with the target trajectory.

In an eight aspect, a method of operating the surgical system of the first aspect is provided.

In a ninth aspect, a method of operating the surgical system of the second aspect is provided.

In a tenth aspect, a method of operating the surgical system of the third aspect is provided.

In an eleventh aspect, a method of operating the surgical system of the fourth aspect is provided.

In a twelfth aspect, a method of operating the surgical system of the fifth aspect is provided.

In a thirteenth aspect, a method of operating the surgical system of the sixth aspect is provided.

Any of the aspects can be combined in part or in whole. Any of the aspects can be combined be in part or in whole with any of the following implementations:

The one or more controllers may be configured to, responsive to the end effector being aligned with the target trajectory, operate the robotic arm in a haptic mode, whereby movement of the end effector is constrained to the target trajectory.

The one or more controllers may be configured to: define a first point and a second point along the target trajectory, wherein the first point is located at a first position along the target trajectory, wherein the second point is located at a second position along the target trajectory, and wherein the first position is further from the anatomy than the second position; responsive to end effector being aligned with the target trajectory, operate the robotic arm in a haptic mode, whereby movement of the end effector is constrained to the target trajectory above the second point; and responsive to movement of the end effector along the target trajectory above the first point during operation of the robotic arm in the haptic mode, cease operation of the robotic arm in the haptic mode. The first point and the second point may be defined based on the tracked pose of the anatomy of the patient in response to the end effector being aligned with the target trajectory. The one or more controllers may be configured to implement haptic feedback to indicate that the end effector has moved above the second point.

The surgical system may comprise an input device, wherein the one or more controllers may be configured to: detect an input from the input device; and in response to detection of the input, operate the robotic arm in the automatic mode to automatically align the end effector with the target trajectory. The one or more controllers the one or more controllers may be configured to: detect an input from the input device; and in response to detection of the input, operate the robotic arm in a haptic mode to constrain movement of the end effector to the target trajectory. The one or more controllers may be configured to implement haptic feedback to indicate that the end effector is aligned with the target trajectory. The one or more controllers may be configured to implement haptic feedback to indicate that the target trajectory is selected. The input device may be defined as a foot pedal. The one or more controllers may be configured to implement haptic feedback to the input device. The one or more controllers may be configured to implement haptic feedback to the end effector.

A pose of the haptic object may be dependent on the tracked pose of the anatomy, and the one or more controllers may be further configured to: responsive to detecting a first input from the input device and responsive to the end effector being aligned with the haptic object, constrain the end effector to the haptic object; and responsive to detecting a second input from the input device and responsive to the end effector being aligned with the haptic object, constrain the end effector independent of the tracked pose of the anatomy. The one or more controllers are configured to constrain the end effector independent of the tracked pose of the anatomy such that an orientation of the end effector does not change when the pose of the haptic object changes. The end effector may extend along an axis, and, to constrain movement of the end effector independent of the tracked pose of the anatomy, the one or more controllers may be configured to constrain the end effector to an orientation of the axis of the end effector at a time of alignment of the axis of the end effector with the haptic object. The one or more controllers may be configured to monitor input from the navigation system to determine the pose of the haptic object. The one or more controllers may be configured to: determine a displacement between the axis of end effector and the haptic object; and evaluate the displacement relative to a realignment threshold. The one or more controllers may be configured to: responsive to a third input and responsive to determining that the displacement is within the realignment threshold, operate the robotic arm in the automatic mode, whereby the robotic arm is automatically moved to align the end effector with the haptic object; and responsive to a fourth input from the input device and responsive to the end effector being aligned with the haptic object, constrain the end effector. The one or more controllers may be configured to, responsive to determining that the displacement exceeds the realignment threshold, cease constraint of the end effector. The first input may be defined as a press of a foot pedal, and the second input may be defined as a release of the foot pedal. The display may be configured to prompt a user to actuate the input device.

The one or more controllers may be configured to: associate a prevention zone with the target trajectory, and responsive to the end effector being within the trajectory selection zone and the prevention zone in the free mode, automatically select the target trajectory associated with the trajectory selection zone. The prevention zone may surround the trajectory selection zone. The prevention zone may be defined as a spherical prevention zone.

The trajectory selection zone may be further defined as a three-dimensional geometry. The three-dimensional geometry may include a rectangular cross-section. The trajectory selection zone may be located at a predetermined distance above a skin surface of the patient.

The target trajectory may be further defined as a first target trajectory, and wherein the one or more controllers are further configured to associate a second target trajectory with the anatomy of the patient. The first target trajectory may extend in a first direction and the second target trajectory may extend in a second direction, and wherein the first direction is different from the second direction. The trajectory selection zone may be further defined as a first trajectory selection zone, and the one or more controllers may be further configured to: associate a second trajectory selection zone with the second target trajectory; and responsive to the end effector being within the second trajectory selection zone in the free mode, automatically select the second target trajectory associated with the second trajectory selection zone. A two-dimensional projection of the first target trajectory and a two-dimensional projection of the second target trajectory may intersect at a point, and the first trajectory selection zone and the second trajectory selection zone may be located at a predetermined distance from the point. A size of the first trajectory selection zone may be equivalent to a size of the second trajectory selection zone.

The anatomy may be defined as including a first and second vertebral body, and the one or more controllers may be configured to: associate the first target trajectory with a right side of the first vertebral body; associate the second target trajectory with the right side of the second vertebral body; associate a third target trajectory with a left side of the first vertebral body; and associate a fourth target trajectory with the left side of the second vertebral body. The one or more controllers may be configured to: define a third trajectory selection zone associated with the third target trajectory; and define a fourth trajectory selection zone associated with the fourth target trajectory.

The one or more controllers may be configured to operate the robotic arm in the automatic mode to align the end effector with the target trajectory by moving the end effector along a tool path, the tool path being based on a point along the target trajectory closest to a position of the end effector.

The surgical system may include a display. The display may be configured to provide a virtual representation of the selected target trajectory, and the display may be configured to highlight the virtual representation of the selected target trajectory. The display may be configured to provide a virtual representation of a planned screw corresponding to the selected target trajectory, and the display may be configured to highlight the virtual representation of the planned screw. The display may be configured to provide a virtual representation of the anatomy associated with the selected target trajectory, and the display may be configured to highlight the virtual representation of the anatomy. The display may be configured to provide a virtual representation of the trajectory selection zone associated with the selected target trajectory, and the display may be configured to highlight the virtual representation of the trajectory selection zone. The display may be configured to indicate that the end effector is aligned with the target trajectory. The one or more controllers may be configured to determine whether the end effector is closer to the first target trajectory or the second target trajectory and the display may be configured to indicate whether the end effector is closer to the first target trajectory or the second target trajectory. The display may be configured to indicate whether the end effector is closer to the first trajectory selection zone or the second trajectory selection zone.

The end effector may include a guide tube configured to support an instrument temporarily affixed to the guide tube. The one or more controllers may be configured to determine whether an instrument is temporarily affixed to the guide tube. The navigation system may be configured to track a pose of an instrument, and the one or more controllers may be configured to determine whether an instrument is temporarily affixed to the guide tube based on a tracked pose of the instrument. The surgical system may further include a sensing system configured to sense an instrument temporarily affixed to the guide tube, and the one or more controllers may be configured to determine whether an instrument is temporarily affixed to the guide tube based on the sensing system sensing that the instrument is temporarily affixed to the guide tube. The one or more controllers may be configured to, responsive to determining that an instrument is temporarily affixed to the guide tube and responsive to the guide tube being aligned with the target trajectory, operate the robotic arm in a haptic mode to constrain movement of the guide tube to the target trajectory and prevent movement of the guide tube away from the anatomy of the patient. The robotic arm may comprise brakes, and the surgical system may include a braking system configured to actuate the brakes to prevent movement of the guide tube away from the anatomy of the patient.

### BRIEF DESCRIPTION OF THE DRAWINGS

Other advantages of the present disclosure will be readily appreciated, as the same becomes better understood by reference to the following detailed description when considered in connection with the accompanying drawings.

FIG. 1 is a perspective view of a robotic surgical system including an end effector.

FIG. 2 is a block diagram of controllers of the robotic surgical system of FIG. 1.

FIG. 3 is a flowchart illustrating a method of aligning an end effector of FIG. 1 with a target trajectory.

FIG. 4 is a diagram illustrating the method of FIG. 3 of aligning an end effector with a target trajectory.

FIGS. 5A-5F are schematic views of a vertebra of a patient and trajectory selection zones defined by the robotic surgical system of FIG. 1.

FIGS. 6A-6C are perspective views of the robotic surgical system of FIG. 1 performing the method of FIG. 3 of aligning an end effector with a target trajectory.

FIG. 7 is an illustration of a display of the robotic surgical system of FIG. 1 providing an indication of alignment of an end effector with a target trajectory.

FIG. 8 is an illustration of a display of the robotic surgical system of FIG. 1 displaying multiple target trajectories.

FIG. 9 is a flowchart illustrating a method of preventing movement of a guide tube away of the end effector of FIG. 1 from an anatomy of a patient.

FIG. 10 is a flowchart illustrating a method of constraining the end effector of FIG. 1.

FIGS. 11A-11C are diagrams illustrating the method of FIG. 10 of constraining the end effector of FIG. 1.

### DETAILED DESCRIPTION

### I. System Overview

With reference to the Figures, wherein like numerals indicate like or corresponding parts throughout the several views, a surgical system 10 (hereinafter “system”) and method for operating the system 10 are described herein and shown throughout the accompanying Figures.

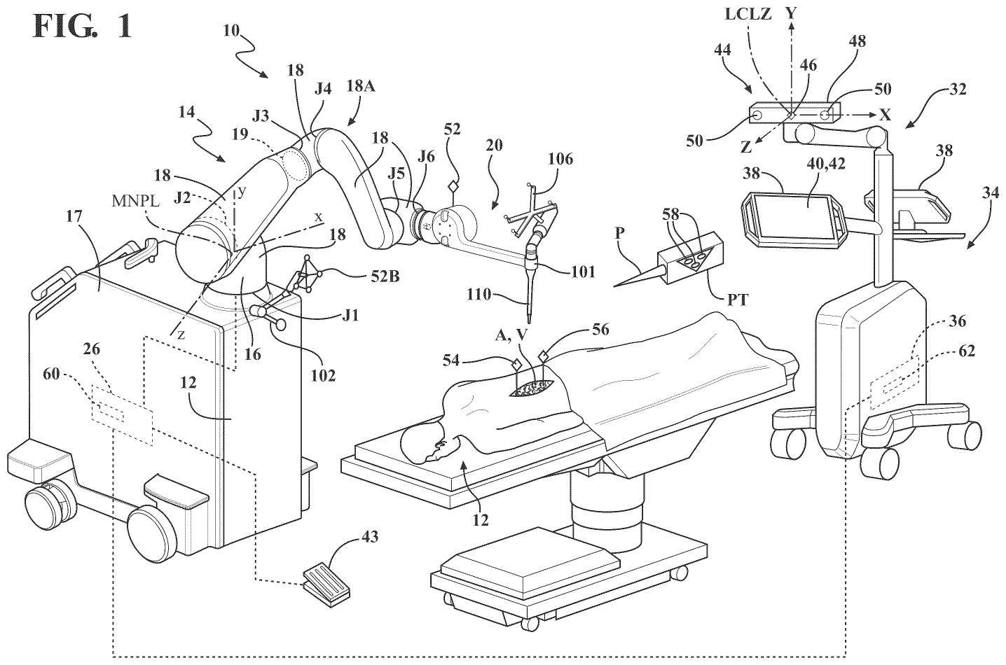

As shown in FIG. 1, the system 10 is a robotic surgical system for treating an anatomy (surgical site) of a patient 12, such as bone or soft tissue. In FIG. 1, the patient 12 is undergoing a surgical procedure. The anatomy A in FIG. 1 includes a spine and vertebra V of the patient 12. The surgical procedure may involve tissue removal or treatment. The robotic surgical system 10 described herein may be utilized for treating any anatomical structure(s), for example, such as joints, including knee joints, hip joints, shoulder joints, ankles joints, or any other bone structure(s) not described herein. The robotic surgical system 10 can be used to perform any type of procedure, including any spinal procedure, partial knee arthroplasty, total knee arthroplasty, total hip arthroplasty, anatomical shoulder arthroplasty, reverse shoulder arthroplasty, fracture repair surgery, osteotomies, and the like. Similarly, the techniques and methods described herein can be used with any type of robotic system and for any procedure.

The system 10 includes a manipulator 14, which may also be referred to as a robotic manipulator. In one example, the manipulator 14 has a base 16 and plurality of links 18. The plurality of links 18 may be commonly referred to as a robotic arm 18A. In some instances, the manipulator 14 may include more than one robotic arm 18A. A manipulator cart 17 supports the manipulator 14 such that the manipulator 14 is fixed to the manipulator cart 17. The links 18 collectively form one or more arms of the manipulator 14. The manipulator 14 may have a serial arm configuration (as shown in FIG. 1) or a parallel arm configuration. In other examples, more than one manipulator 14 may be utilized in a multiple arm configuration. The manipulator 14 comprises a plurality of joints (J) and a plurality of joint encoders 19 located at the joints (J) for determining position data of the joints (J). For simplicity, one joint encoder 19 is illustrated in FIG. 1, although it is to be appreciated that the other joint encoders 19 may be similarly illustrated. The manipulator 14 according to one example has six joints (J1-J6) implementing at least six-degrees of freedom (DOF) for the manipulator 14. However, the manipulator 14 may have any number of degrees of freedom and may have any suitable number of joints (J) and redundant joints (J). In one example, each of the joints (J) of the manipulator 14 are actively driven and may be motorized joints (J). In other examples, each of the joints (J) may be passively driven. In still other examples, the joints (J) may include a combination of actively driven joints (J) and passively driven joints (J).

The base 16 of the manipulator 14 is generally a portion of the manipulator 14 that is stationary during usage thereby providing a fixed reference coordinate system (i.e., a virtual zero pose) for other components of the manipulator 14 or the system 10 in general. Generally, the origin of a base coordinate system is defined at the fixed reference of the base 16. The base coordinate system may be referred to herein as a manipulator coordinate system MNPL and the robotic arm 18A is configured to support and move an end effector coupled to the robotic arm 18A in the manipulator coordinate system MNPL. The fixed reference point of the base 16 may be defined with respect to any suitable portion of the manipulator 14, such as one or more of the links 18. Alternatively, or additionally, the fixed reference point of the base 16 may be defined with respect to the manipulator cart 17, such as where the manipulator 14 is physically attached to the cart 17. In one example, the fixed reference point of the base 16 is defined at an intersection of the axes of joints J1 and J2. Thus, although joints J1 and J2 are moving components in reality, the intersection of the axes of joints J1 and J2 is nevertheless a virtual fixed reference point, which does not move in the manipulator coordinate system MNPL. The manipulator 14 and/or manipulator cart 17 house a manipulator computer 26, or other type of control unit.

The system 10 may include an end effector 20 coupled to the robotic arm 18A. The end effector 20 may include any end effector suitable for a surgical procedure. In some instances, the end effector 20 may include a surgical instrument such that the surgical instrument is supported by the robotic arm 18A. The surgical instrument may be any instrument for manipulating the anatomy A of a patient, such as a saw, a cutting burr, a router, a reamer, an impactor, an ultrasonic aspirator, a probe, a scalpel, a trocar, a cutting tool, a drill, a dilator, a screwdriver, an intervertebral inserter, a distractor, an abrader, a discectomy tool, or the like. In the instance of FIG. 1, the end effector 20 includes a surgical instrument 110, which is illustrated as a drill device. Additionally, or alternatively, the end effector 20 may include an accessory and/or energy applicator, such as a saw blade, a cutting burr, a router, a reamer, an impactor, an ultrasonic aspirator, a probe, a scalpel, a trocar, a cutting tool, a drill, a dilator, a screwdriver, an intervertebral inserter, a distractor, an abrader, a discectomy tool, or the like. The accessory and energy applicator may be integrated or separately attached to the end effector 20. The end effector 20 may also include a cutting guide. As shown in FIG. 1, the end effector 20 may include a tool holder, which may support any of the surgical instruments described above. The tool holder may be a guide tube 101 for supporting a surgical instrument that can be temporarily affixed to the guide tube 101 and/or slidable within the guide tube 101. The guide tube 101 may be the guide tube further described in U.S. Provisional Patent Application No. 63/612,011, entitled, “Magnetic Spine Registration Tool”, which is incorporated herein by reference. Additionally, the guide tube 101 may the anti-skiving guide tube described in U.S. Provisional Patent Application No. 63/454,346, entitled, “Anti-Skiving Guide Tube And Surgical System Including The Same”, which is incorporated herein by reference. Additionally, or alternatively, the surgical instruments can be actively driven or motorized by the robotic manipulator 14. The surgical instruments can be hand-held and selectively coupled to the robotic manipulator 14.

The system 10 may include one or more tool trackers 106. The tool tracker 106 may be temporarily coupled to the end effector 20. For example, the tool tracker 106 may be the trackable array described in U.S. Pat. App. Pub. No. 2022/0134569, entitled, “Robotic Surgical System With Motorized Movement To A Starting Pose For A Registration Or Calibration Routine,” the disclosure of which is hereby incorporated by reference, or such as the end effector tracker described in U.S. Pat. No. 10,350,012, entitled, “Method And Apparatus For Controlling A Haptic Device,” the disclosure of which is hereby incorporated by reference, or such as the tool tracker in U.S. Provisional Patent Application No. 63/612,011, entitled, “Magnetic Spine Registration Tool”, which is incorporated herein by reference. In other examples, the tool tracker 106 may be attachable to or detachable from the end effector 20 and/or attachable to or detachable from any other component of the manipulator 14, such as one or more links of the robotic arm 18A, e.g. a distal-most link of the manipulator (J6). For instance, the tool tracker 106 may include similar components as the tracker assembly described in U.S. Pat. App. Pub. No. 2023/0277256, entitled, “Robotic System Including A Link Tracker,” the disclosure of which is hereby incorporated by reference, for attaching the tool tracker 106 to the end effector 20 or any other component of the manipulator 14. For instance, the tool tracker 106 may be attached/detached to the end effector 20 or any other component of the manipulator 14 using a spring-biased latch, a magnetic connection, a snap-fit connection using flexible elements, or the like. In other examples, the tool tracker 106 may be temporarily coupled to the end effector 20 via a component of the end effector 20. For example, in instances where the end effector 20 includes the surgical instrument 110, such as the instance of FIG. 1, the tool tracker 106 may be coupled to the end effector 20 via the surgical instrument 110. As another example, in instances where the end effector 20 includes the guide tube 101, the tool tracker 106 may be coupled to the end effector 20 via the guide tube 101. Additionally, the system 10 may include more than one tool tracker 106. For example, in instances where the end effector 20 includes the guide tube 101 configured to support a surgical instrument 110 temporarily affixed to the guide tube 101, a first tool tracker 106 may be coupled to the guide tube 101 and a second tool tracker 106 may be coupled to the surgical instrument 110.

The tool tracker 106 may be coupled to the end effector 20 such that a relationship between the tool tracker 106 and the end effector 20 may be determinable. For example, the tool tracker 106 may include a reference surface configured to abut the end effector 20, such as the reference surface described in U.S. Provisional Patent Application No. 63/612,011, entitled, “Magnetic Spine Registration Tool”, which is incorporated herein by reference. Contact between the reference surface and the end effector 20 may indicate that the tool tracker 106 is properly coupled to the end effector 20 such that a location of the tool tracker 106 relative to the end effector 20 is fixed and that a relationship between the tool tracker 106 and the end effector 20 is determinable.

The tool tracker 106 may include one or more fiducial markers FM. In some instances, the fiducial markers FM may be coupled to or integrally formed with or manually coupled to the end effector 20 and/or a component of the manipulator 14. The fiducial markers FM may include any suitable shape. For example, the fiducial markers FM may include a cuboidal or elliptical shape. The fiducial markers FM may be active or passive tracking elements.

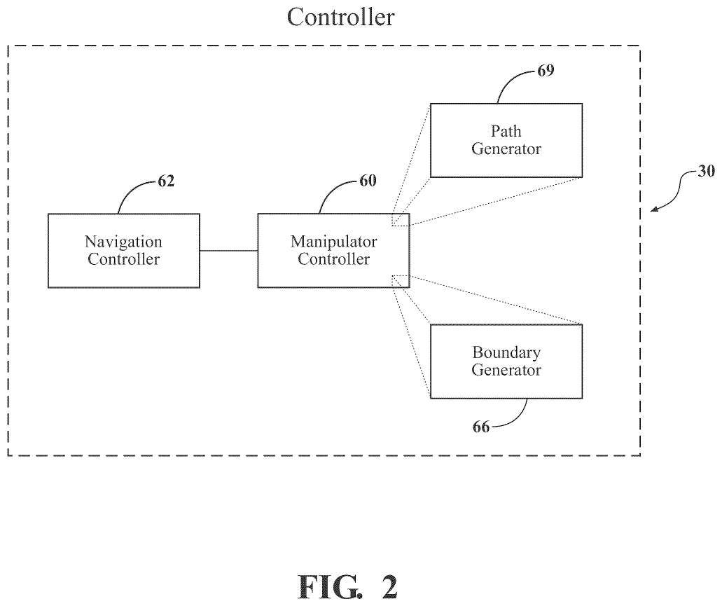

Referring to FIG. 2, the system 10 includes one or more controllers 30 (hereinafter referred to as “controller”). The controller 30 includes software and/or hardware for controlling the manipulator 14. The controller 30 directs the motion of components of the manipulator 14, such as the robotic arm 18A, and controls a pose (position and/or orientation) of the end effector 20 with respect to a coordinate system of the robotic arm 18A. In one example, the coordinate system of the robotic arm 18A is the manipulator coordinate system MNPL, as shown in FIG. 1, and the controller 30 may be configured to control the robotic arm 18A to the robotic arm 18A to support and move the end effector 20 in the manipulator coordinate system MNPL. The manipulator coordinate system MNPL has an origin located at any suitable pose with respect to the manipulator 14. Axes of the manipulator coordinate system MNPL may be arbitrarily chosen as well. Generally, the origin of the manipulator coordinate system MNPL is defined at the fixed reference point of the base 16. One example of the manipulator coordinate system MNPL is described in U.S. Pat. No. 9,119,655, entitled, “Surgical Manipulator Capable of Controlling a Surgical Instrument in Multiple Modes,” the disclosure of which is hereby incorporated by reference.

As shown in FIG. 1, the system 10 further includes a navigation system 32. One example of the navigation system 32 is described in U.S. Pat. No. 9,008,757, filed on Sep. 24, 2013, entitled, “Navigation System Including Optical and Non-Optical Sensors,” hereby incorporated by reference. The navigation system 32 is configured to track movement of various objects. Such objects include, for example, the manipulator 14, the end effector 20, and/or the anatomy A. The navigation system 32 tracks these objects to gather state information of one or more of the objects with respect to a (navigation) localizer coordinate system LCLZ. Coordinates in the localizer coordinate system LCLZ may be transformed to the manipulator coordinate system MNPL, and/or vice-versa, using transformation and registration techniques described in U.S. Provisional Patent Application No. 63/552,897, entitled “Systems and Method for Image Based Registration and Calibration,” U.S. Provisional Patent Application No. 63/612,011, entitled, “Magnetic Spine Registration Tool”, and U.S. patent application Ser. No. 17/513,324, entitled “Robotic Surgical System with Motorized Movement to a Starting Pose for a Registration or Calibration Routine”, which are incorporated herein by reference.

The navigation system 32 can include a cart assembly 34 that houses a navigation computer 36, and/or other types of control units. A navigation interface is in operative communication with the navigation computer 36. The navigation interface includes one or more displays 38. The navigation system 32 is capable of displaying a graphical representation of the relative states of the tracked objects to the operator using the one or more displays 38.

The navigation system 32 is configured to depict a visual representation of the anatomy A and the manipulator 14, and/or end effector 20 for visual guidance of any of the techniques described. The visual representation may be real (camera) images, virtual representations (e.g., computer models), or any combination thereof. The visual representation can be presented on any display viewable to the surgeon, such as the displays 38 of the navigation system 32, head mounted devices, or the like. The representations may be augmented reality, mixed reality, or virtual reality.

The navigation system 32 also includes a navigation localizer 44 (hereinafter “localizer”) coupled to the navigation computer 36. In one example, the localizer 44 is an optical localizer and includes a camera unit 46. The camera unit 46 has an outer casing 48 that houses one or more optical sensors 50.

The navigation system 32 may include one or more trackers, which may be tracked by the localizer 44. In one example, the trackers include the tool tracker 106, a pointer tracker PT, one or more manipulator trackers 52, and/or one or more patient trackers 54, 56. In the illustrated example of FIG. 1, the manipulator tracker 52 is attached to a distal flange of the robotic arm 18A. The manipulator tracker 52 may be affixed to any suitable component of the manipulator 14, in addition to, or other than the surgical tool, such as the base 16 (i.e., tracker 52B), or any one or more links 18 or joints J of the manipulator 14. Additionally, or alternatively, the manipulator tracker 52 may be secured to a surgical drape or drape assembly, as described in U.S. Pat. App. Pub. No. 2023/0277256, entitled, “Robotic System Including A Link Tracker,” the disclosure of which is hereby incorporated by reference. For instance, the manipulator tracker 52 may be secured to a surgical drape or drape assembly via an elastic band or snap ring. The patient trackers may be affixed to a vertebra V of the patient 12 and/or the pelvis of the patient 12. In the illustrated example of FIG. 1, the first patient tracker 54 is firmly affixed to a vertebra V of the patient 12, and the second patient tracker 56 is firmly affixed to pelvis of the patient 12. In this example, the patient trackers 54, 56 are firmly affixed to sections of bone. The pointer tracker PT is firmly affixed to a pointer P used for registering the anatomy A to the localizer coordinate system LCLZ. Those skilled in the art appreciate that the trackers described herein may be fixed to their respective components in any suitable manner.

As shown in FIG. 1, the base tracker 52B may be coupled to the cart 17 by an adjustable support arm 102. As shown, the base tracker 52B may be attached to one end of an adjustable support arm 102 and the adjustable support arm 102 may be attached at the other end to the cart 17. The adjustable support arm 102 can be positioned and locked to place the base tracker 52B in a fixed position relative to the cart 17. An example of a base tracker 52B coupled to an adjustable support arm can be like that described in U.S. patent application Ser. No. 17/513,324, entitled, “Robotic Surgical System With Motorized Movement To A Starting Pose For A Registration Or Calibration Routine”, or U.S. patent application Ser. No. 18/198,938, entitled, “Robotic System With Improved Configurations For Base Tracker”, the entire contents of which are hereby incorporated by reference in their entirety. Alternatively, or additionally, a base tracker 52B may be coupled to the robotic arm 18A and may be moveable with the robotic arm 18A. For instance, the base tracker 52B may include a plurality of (active or passive) tracking elements located on any number of links 18 of the manipulator 14. In this case, the base tracker 52B is formed of a tracking geometry from the various tracking elements, which move with movement of the robotic arm 18A. An example of a base tracker 52B formed by optical markers located on the links 18 may be like that described in U.S. patent application Ser. No. 18/115,964, entitled, “Robotic System with Link Tracker”, the entire contents of which is hereby incorporated by reference in its entirety. Alternatively, or additionally, the base tracker 52B may be secured to a surgical drape or drape assembly, as described in U.S. Pat. App. Pub. No. 2023/0277256, entitled, “Robotic System Including A Link Tracker,” the disclosure of which is hereby incorporated by reference. For instance, the base tracker 52B may be secured to a surgical drape or drape assembly via an elastic band or snap ring.

When optical localization is utilized, however, one or more of the trackers may include active markers 58. The active markers 58 may include light emitting diodes (LEDs). Alternatively, the trackers described herein may have passive markers, such as reflectors, which reflect light emitted from the camera unit 46. Other suitable markers not specifically described herein may be utilized.

The localizer 44 tracks the trackers to determine a state of one or more of the trackers which correspond respectively to the state of the object respectively attached thereto. The localizer 44 provides the state of the trackers to the navigation computer 36. In one example, the navigation computer 36 determines and communicates the state the trackers to the manipulator computer 26. As used herein, the state of an object includes, but is not limited to, data that defines the position and/or orientation of the tracked object or equivalents/derivatives of the position and/or orientation. For example, the state may be a pose of the object, and may include linear data, and/or angular velocity data, and the like.

Although one example of the navigation system 32 is shown in the Figures, the navigation system 32 may have any other suitable configuration for tracking the manipulator 14 and the patient 12. The illustrated tracker configuration is provided merely as one example for tracking objects within the operating space. Any number of trackers may be utilized and may be located in positions or on objects other than shown. In other examples, such as described below, the localizer 44 may detect objects absent any trackers affixed to objects.

In one example, the navigation system 32 and/or localizer 44 are ultrasound-based. For example, the navigation system 32 may comprise an ultrasound imaging device coupled to the navigation computer 36. The ultrasound imaging device may be robotically controlled or may be hand-held. The ultrasound imaging device images any of the aforementioned objects, e.g., the manipulator 14 and the patient 12, and generates state signals to the controller 30 based on the ultrasound images. The ultrasound images may be of any ultrasound imaging modality. The navigation computer 36 may process the images in near real-time to determine states of the objects. Ultrasound tracking can be performed absent the use of trackers affixed to the objects being tracked. The ultrasound imaging device may have any suitable configuration and may be different than the camera unit 46 as shown in FIG. 1. One example of an ultrasound tracking system can be like that described in U.S. patent application Ser. No. 15/999,152, filed Aug. 16, 2018, entitled “Ultrasound Bone Registration With Learning-Based Segmentation And Sound Speed Calibration,” the entire contents of which are incorporated by reference herein.

In another example, the navigation system 32 and/or localizer 44 are radio frequency (RF)-based. For example, the navigation system 32 may comprise an RF transceiver coupled to the navigation computer 36. The manipulator 14 and the patient 12 may comprise RF emitters or transponders attached thereto. The RF emitters or transponders may be passive or actively energized. The RF transceiver transmits an RF tracking signal and generates state signals to the controller 30 based on RF signals received from the RF emitters. The navigation computer 36 and/or the controller 30 may analyze the received RF signals to associate relative states thereto. The RF signals may be of any suitable frequency. The RF transceiver may be positioned at any suitable location to track the objects using RF signals effectively. Furthermore, the RF emitters or transponders may have any suitable structural configuration that may be much different than the trackers as shown in FIG. 1.

In yet another example, the navigation system 32 and/or localizer 44 are electromagnetically based. For example, the navigation system 32 may comprise an EM transceiver coupled to the navigation computer 36. The manipulator 14 and the patient 12 may comprise EM components attached thereto, such as any suitable magnetic tracker, electro-magnetic tracker, inductive tracker, or the like. The trackers may be passive or actively energized. The EM transceiver generates an EM field and generates state signals to the controller 30 based upon EM signals received from the trackers. The navigation computer 36 and/or the controller 30 may analyze the received EM signals to associate relative states thereto. Again, such navigation system 32 examples may have structural configurations that are different than the navigation system 32 configuration as shown throughout the Figures.

In yet another example, the navigation system 32 and/or localizer 44 utilize a machine vision system which includes a video camera coupled to the navigation computer 36. The video camera is configured to locate a physical object in a target space. The physical object has a geometry represented by virtual object data stored by the navigation computer 36. The detected objects may be tools, obstacles, anatomical features, trackers, or the like. The video camera and navigation computer 36 are configured to detect the physical objects using image processing techniques such as pattern, color, or shape recognition, edge detection, pixel analysis, neutral net or deep learning processing, optical character recognition, barcode detection, or the like. The navigation computer 36 can compare the captured images to the virtual object data to identify and track the objects. A tracker may or may not be coupled to the physical object. If trackers are utilized, the machine vision system may also include infrared detectors for tracking the trackers and comparing tracking data to machine vision data. Again, such navigation system 32 examples may have structural configurations that are different than the navigation system 32 configuration as shown throughout the Figures. Examples of machine vision tracking systems can be like that described in U.S. Pat. No. 9,603,665, entitled “Systems and Methods for Establishing Virtual Constraint Boundaries” and/or like that described in U.S. Provisional Patent Application No. 62/698,402, filed Jul. 16, 2018, entitled “Systems and Method for Image Based Registration and Calibration,” the entire contents of which are incorporated by reference herein.

The navigation system 32 and/or localizer 44 may have any other suitable components or structure not specifically recited herein. Furthermore, any of the techniques, methods, and/or components described above with respect to the camera-based navigation system 32 shown throughout the Figures may be implemented or provided for any of the other examples of the navigation system 32 described herein. For example, the navigation system 32 may utilize solely inertial tracking or any combination of tracking techniques.

As shown in FIG. 2, the controller 30 further includes software modules. The software modules may be part of a computer program or programs that operate on the manipulator computer 26, navigation computer 36, or a combination thereof, to process data to assist with control of the system 10. The software modules include instructions stored in one or more non-transitory computer readable medium or memory on the manipulator computer 26, navigation computer 36, or a combination thereof, to be executed by one or more processors of the computers 26, 36. Additionally, software modules for prompting and/or communicating with the operator may form part of the program or programs and may include instructions stored in memory on the manipulator computer 26, navigation computer 36, or a combination thereof.

The controller 30 includes a manipulator controller 60 for processing data to direct motion of the manipulator 14. In one example, as shown in FIG. 1, the manipulator controller 60 is implemented on the manipulator computer 26. The manipulator controller 60 may receive and process data from a single source or multiple sources. The controller 30 further includes a navigation controller 62 for communicating the state data relating to the anatomy A to the manipulator 14 to the manipulator controller 60. The manipulator controller 60 receives and processes the state data provided by the navigation controller 62 to direct movement of the manipulator 14. In one example, as shown in FIG. 1, the navigation controller 62 is implemented on the navigation computer 36. The manipulator controller 60 or navigation controller 62 may also communicate states of the patient 12 and manipulator 14 to the operator by displaying an image of the anatomy A and the manipulator 14 on the one or more displays 38. The manipulator computer 26 or navigation computer 36 may also command display of instructions or request information using the display 38 to interact with the operator and for directing the manipulator 14.

The system 10 may include one or more input devices 40, 42, 43 for receiving an input from an operator interacting with an input device 40, 42, 43. In FIG. 1, the first and second input devices 40, 42 are shown as interactive touchscreen displays and the third input device 43 is shown as a footswitch including a user-actuatable foot pedal. In other instances, the input devices 40, 42, 43 may be any device for receiving an input from an operator. For example, the input device 40, 42, 43 may include any one or more of a keyboard, a mouse, a remote-control device, a microphone (voice-activation), gesture control devices, head-mounted devices, and the like.

Additionally, it is contemplated that the operator may interact with the input devices 40, 42, 43 in any suitable manner and the input devices 40, 42, 43 may receive a corresponding input. For example, the operator may interact with the input devices 40, 42, 43 by pressing, holding, clicking, double-clicking, releasing, and/or performing any other suitable interaction with an input of the input devices 40, 42, 43. In a more specific instance, the operator may interact with the footswitch 43 of FIG. 1 by pressing, holding, clicking, double-clicking, and/or releasing the user-actuatable foot pedal of the footswitch 43. Similarly, the operator may interact with the interactive touchscreen displays 40, 42 of FIG. 1 by pressing, holding, clicking, double-clicking, swiping, and/or releasing a portion of a graphical user interface of the interactive touchscreen displays 40, 42.

The input devices 40, 42, 43 may be coupled to the controller 30 and the controller 30 may detect an input from the input devices 40, 42, 43. For example, the detected input may be a detected actuation of the footswitch 43 or a detected interaction with an interactive touchscreen display 40, 42. In this way, the operator may interact with the input devices 40, 42, 43 to communicate with the software modules shown in FIG. 2. For example, the input devices 40, 42, 43 may be actuated by an operator to input information into and/or select/control certain aspects of the manipulator controller 60, the manipulator computer 26, the navigation controller 62, and/or the navigation computer 36. The input devices 40, 42, 43 may be configured to communicate with the controller 30 via a user interface software. The user interface software may run on the manipulator computer 26 and navigation computer 36, or on a separate device from the manipulator computer 26 and navigation computer 36.

The controller 30, including the manipulator controller 60 and navigation controller 62, may be implemented on any suitable device or devices in the system 10, including, but not limited to, the manipulator computer 26, the navigation computer 36, and any combination thereof. As will be described herein, the controller 30 is not limited to one controller, but may include a plurality of controllers for various systems, components, or sub-systems of the surgical system 10. These controllers may be in communication with each other (e.g., directly, or indirectly), and/or with other components of the surgical system 10, such as via physical electrical connections (e.g., a tethered wire harness) and/or via one or more types of wireless communication (e.g., with a WiFi™ network, Bluetooth®, a radio network, and the like). Any of the controller 30 may be realized as or with various arrangements of computers, processors, control units, and the like, and may comprise discrete components or may be integrated (e.g., sharing hardware, software, inputs, outputs, and the like). Any of the one or more controllers may implement their respective functionality using hardware-only, software-only, or a combination of hardware and software. Examples of hardware include, but is not limited, single or multi-core processors, CPUs, GPUs, integrated circuits, microchips, or ASICs, digital signal processors, microcontrollers, field programmable gate arrays, systems on a chip, discrete circuitry, and/or other suitable hardware, and the like. The one or more controllers may implement software programs, software modules, algorithms, logical rules, look-up tables and other reference data, and various software layers for implementing any of the capabilities described herein. Equivalents of the software and hardware for the controller 30, and peripheral devices connected thereto, are fully contemplated.

As shown in FIG. 2, the controller 30 includes a boundary generator 66. The boundary generator 66 is a software module that may be implemented on the manipulator controller 60. Alternatively, the boundary generator 66 may be implemented on other components, such as the navigation controller 62. The boundary generator 66 generates haptic objects for constraining the manipulator 14 and/or the end effector 20. Such haptic objects may include virtual boundaries (VB), cutting planes, target trajectories, virtual meshes, virtual constraints, or the like. The haptic objects may be defined with respect to a 3-D bone model registered to one or more patient trackers such that the haptic objects are fixed relative to the bone model. The state of the manipulator 14 and/or the end effector 20 is tracked relative to the haptic objects. In one example, the state of a center point of the end effector 20 is measured relative to the haptic objects for purposes of determining when and where haptic feedback force is applied to the manipulator 14, or more specifically, the end effector 20.

The haptic object generated by the boundary generator 66 may a target trajectory. In such instances, the controller 30 may align the end effector 20 with the target trajectory and constrain movement of the manipulator 14 and/or end effector 20 to the target trajectory. The boundary generator 66 may define the target trajectory based on a 3-D bone model and associate the target trajectory with the 3-D bone model. For example, the target trajectory may be a desirable trajectory for drilling into a bone of a patient and/or a desirable trajectory for inserting a pedicle screw into a bone of a patient. Additionally, the boundary generator 66 may define and associate more than one target trajectory for a 3-D bone model. For example, in an instance where the boundary generator 66 defines a target trajectory for a drilling into and inserting a pedicle screw into a vertebra of a patient, the boundary generator 66 may generate right side and left side trajectories for drilling into and inserting pedicle screws into a right side and left side of the vertebra, respectively. The boundary generator 66 may generate right side and left side trajectories for each vertebra of the spine of the patient 12.

The controller 30 may be configured to provide haptic and/or audible feedback to an operator of the surgical system 10. The controller 30 may provide haptic and/or audible feedback to an operator to provide the operator with guidance based on virtual boundaries (VB), such as a haptic object, and/or to provide a notification/indication to the operator. The controller 30 may implement haptic and/or audible feedback to any suitable component of the surgical system. For example, the controller 30 may implement haptic feedback to one or more of the input devices 40, 42, 43, to the end effector 20, and/or to the robotic arm 18A. As another example, the controller 30 may implement haptic feedback via a haptic device, such as the haptic device described in described in U.S. Pat. No. 10,350,012, entitled, “Method And Apparatus For Controlling A Haptic Device,” the disclosure of which is hereby incorporated by reference. Additionally, the controller 30 may implement haptic feedback via a speaker of the surgical system 10, such as a speaker of the input devices 40, 42, 43.

Furthermore, the haptic and/or audible feedback may be any suitable feedback means. For example, the haptic feedback may be a vibration pattern or a button-clicking sensation provided to a component of the surgical system, such as one or more of the input devices 40, 42, 43. The audible feedback may be any sound provided by a component of the surgical system, such as one or more of the input devices 40, 42, 43. For instance, the sound may be a button-clicking sound.

A tool path generator 68 is another software module run by the controller 30, and more specifically, the manipulator controller 60. The tool path generator 68 generates a path for the manipulator 14 and/or the end effector 20 to traverse, such as for removing sections of the anatomy A to receive an implant. One exemplary system and method for generating the tool path is explained in U.S. Pat. No. 9,119,655, entitled, “Surgical Manipulator Capable of Controlling a Surgical Instrument in Multiple Modes,” the disclosure of which is hereby incorporated by reference. In some examples, the virtual boundaries (VB) and/or tool paths may be generated offline rather than on the manipulator computer 26 or navigation computer 36. Thereafter, the virtual boundaries (VB) and/or tool paths may be utilized at runtime by the manipulator controller 60.

Additionally, it may be desirable to control the manipulator 14 in different modes of operation for the system 10. For example, the controller 30 may control the manipulator 14/robotic arm 18A to interact with the site using semi-autonomous, automatic, manual/free, and haptic modes of operation.

In the semi-autonomous and automatic modes, the controller 30 directs movement of the robotic arm 18A and/or the end effector 20 at the surgical site. In one instance, the controller 30 models the robotic arm 18A and/or the end effector 20 as a virtual rigid body and determines forces and torques to apply to the virtual rigid body to advance and constrain the robotic arm 18A and/or the end effector 20 along any trajectory or path in the semi-autonomous and automatic modes. Movement of the tool 20 in the semi-autonomous and automatic modes is constrained in relation to the virtual constraints generated by the boundary generator 66 and/or the path generator 69.

In the semi-autonomous mode, the controller 30 are capable of moving the robotic arm 18A and/or end effector 20 free of operator assistance. Free of operator assistance may mean that an operator does not physically move the robotic arm 18A and/or end effector 20 by applying external force to move the robotic arm 18A and/or end effector 20. Instead, the operator may use some form of control to manage starting and stopping of movement. For example, the operator may hold down a button of a control to start movement of the robotic arm 18A and/or end effector 20 and release the button to stop movement of the robotic arm 18A and/or end effector 20. Alternatively, the operator may press a button to start movement of the robotic arm 18A and/or end effector 20 and press a button to stop motorized movement of the robotic arm 18A and/or end effector 20 along the trajectory or path. The controller 30 uses motorized movement to advance the robotic arm 18A and/or end effector 20 in accordance with pre-planned parameters. An example of the semi-autonomous mode is described in U.S. Pat. No. 9,119,655, entitled, “Surgical Manipulator Capable of Controlling a Surgical Instrument in Multiple Modes,” the disclosure of which is hereby incorporated by reference.

In the manual/free mode, the robotic arm 18A is freely moveable. In some instances, the operator manually directs, and the controller 30 control, movement of the robotic arm 18A and/or end effector 20 at the surgical site. For instance, the operator may physically contact the robotic arm 18A and/or end effector 20 to direct movement of the robotic arm 18A and/or end effector 20. In one implementation, the controller 30 may monitor the forces and torques placed on the robotic arm 18A and/or end effector 20 by the operator to position the robotic arm 18A and/or end effector 20. A sensor that is part of the manipulator 14, such as a force-torque transducer, measures these external forces and torques applied to the robotic arm 18A and/or end effector 20, e.g., in six degrees of freedom. In one example, the sensor is coupled between the distal-most link of the manipulator (J6) and the robotic arm 18A and/or end effector 20. In response to the applied forces and torques, the controller 30 are configured to determine a commanded position of the robotic arm 18A and/or end effector 20 by evaluating the forces/torques applied externally to the robotic arm 18A and/or end effector 20 with respect to virtual model of the robotic arm 18A and/or end effector 20 in a virtual simulation. The controller 30 then mechanically move the robotic arm 18A and/or end effector 20 to the commanded position in a manner that emulates the movement that would have occurred based on the forces and torques applied externally by the operator. In instances where the joints (J) are passively driven, the operator may apply force to the robotic arm 18A and/or end effector 20 to cause displacement of the robotic arm 18A and/or end effector 20, without the controller 30 monitoring the forces and torques placed on the robotic arm 18A and/or end effector 20 by the operator. Movement of the robotic arm 18A and/or end effector 20 in the manual/free mode may also constrained in relation to the virtual constraints generated by the boundary generator 66 and/or path generator 69.

The surgical system 10 may be configured to operate in the haptic mode while operating in the manual/free mode. In the haptic mode, the surgical system 10 provides haptic force feedback to an operator in response to movement of the robotic arm 18A and/or end effector 20 at the surgical site. For example, in one implementation, the operator applies force to cause displacement of the robotic arm 18A and/or end effector 20 and the surgical system 10 in the manual/free mode and the surgical system 10 can reactively provide haptic force feedback when the robotic arm 18A and/or end effector 20 reaches certain virtual constraints generated by the boundary generator 66 and/or the path generator 69. In one instance, the haptic force feedback may be provided to the operator via the robotic arm 18A and/or end effector 20. In another instance, the haptic force feedback may be provided to the operator via one or more of the input devices 40, 42, 43.

### II. Aligning the End Effector to a Target Trajectory

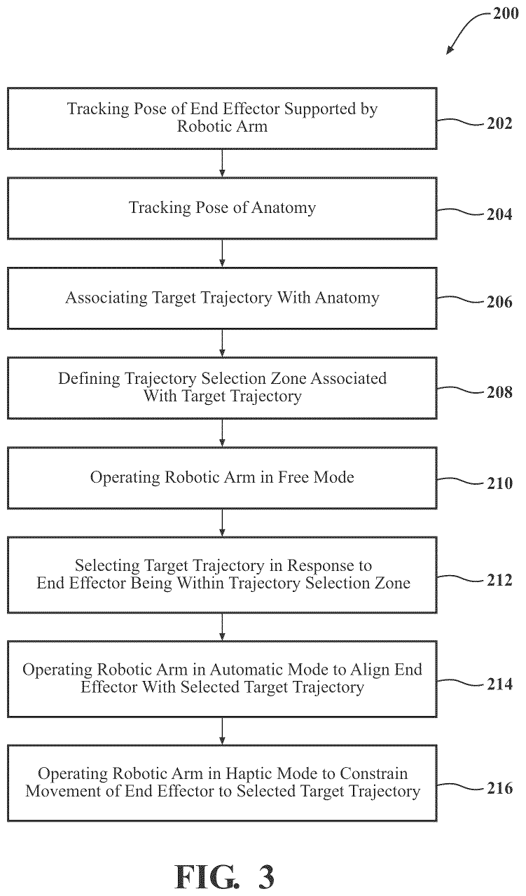

The surgical system 10 may provide a method 200 of aligning the end effector 20 to a target trajectory, shown in FIG. 3. As shown, the method 200 includes a step 202 of tracking a pose of the end effector 20 supported by the robotic arm 18A; a step 204 of tracking a pose of an anatomy A of a patient 12; a step 206 of associating a target trajectory with the anatomy A of the patient 12; a step 208 of defining a trajectory selection zone associated with the target trajectory; a step 210 of operating the robotic arm 18A in the free mode; a step 212 of selecting a target trajectory in response to the end effector 20 being within the associated trajectory selection zone; a step 214 of operating the robotic arm 18A in the automatic mode to align the end effector 20 with the selected target trajectory; and a step 216 of operating the robotic arm 18A in the haptic mode to constrain movement of the end effector 20 to the selected target trajectory T.

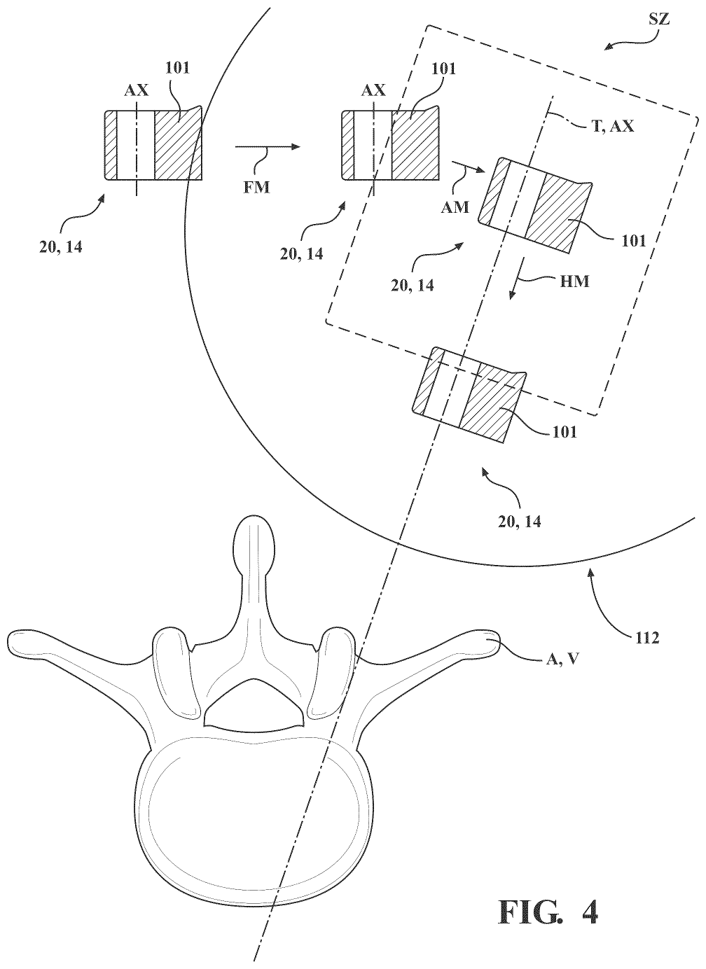

Generally, the method 200 of aligning the end effector 20 to a target trajectory T is shown in FIG. 4. In the instance of FIG. 4, the target trajectory T is associated with a vertebra V of the patient 12 and a trajectory selection zone SZ is defined and associated with the target trajectory T. Additionally, the end effector 20 includes a guide tube 101 extending along an axis AX. Initially, the manipulator 14 may operate in the free mode, allowing the guide tube 101 to be freely moved toward a trajectory selection zone SZ. Movement of the guide tube 101 in the free mode is represented using the arrow FM. Once the guide tube 101 is moved to be within the trajectory selection zone SZ, the target trajectory T is selected and the manipulator 14 may operate in the automatic mode to automatically align the guide tube 101 with the selected target trajectory T. Movement of the guide tube 101 in the automatic mode is represented using the arrow AM. Once the guide tube 101 is aligned with the selected target trajectory T, the manipulator 14 may operate in the haptic mode whereby the guide tube 101 is constrained to the selected target trajectory T. Movement of the guide tube 101 in the haptic mode is represented using the arrow HM.

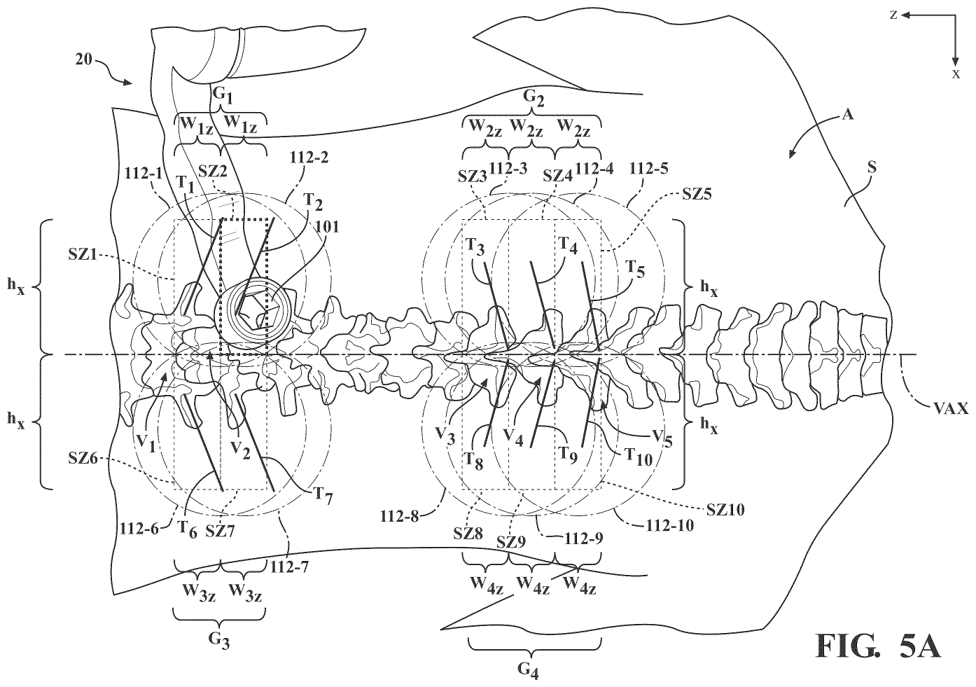

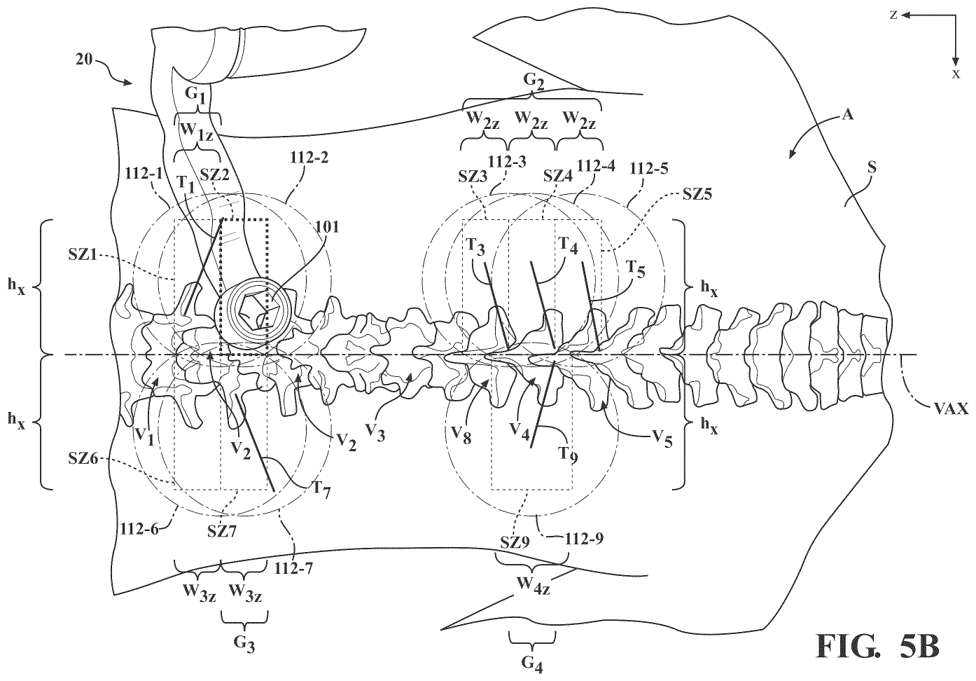

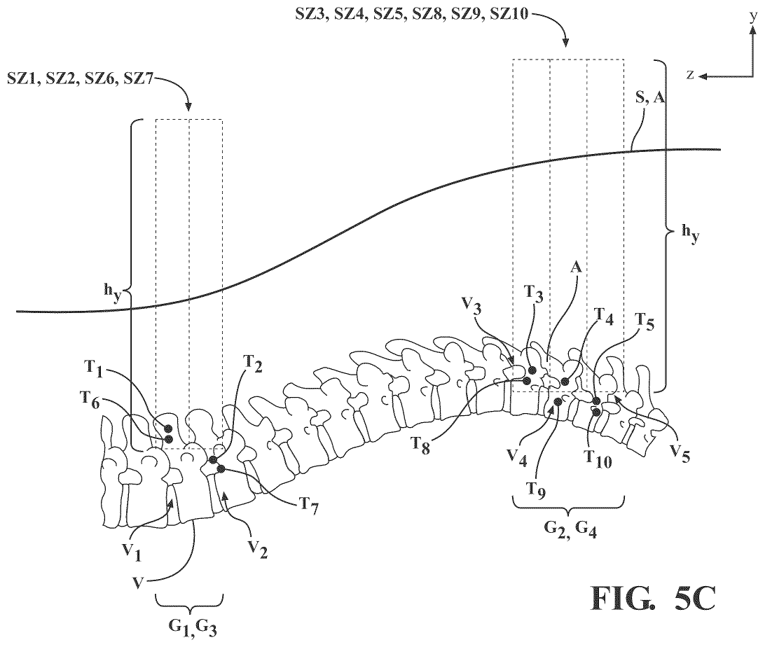

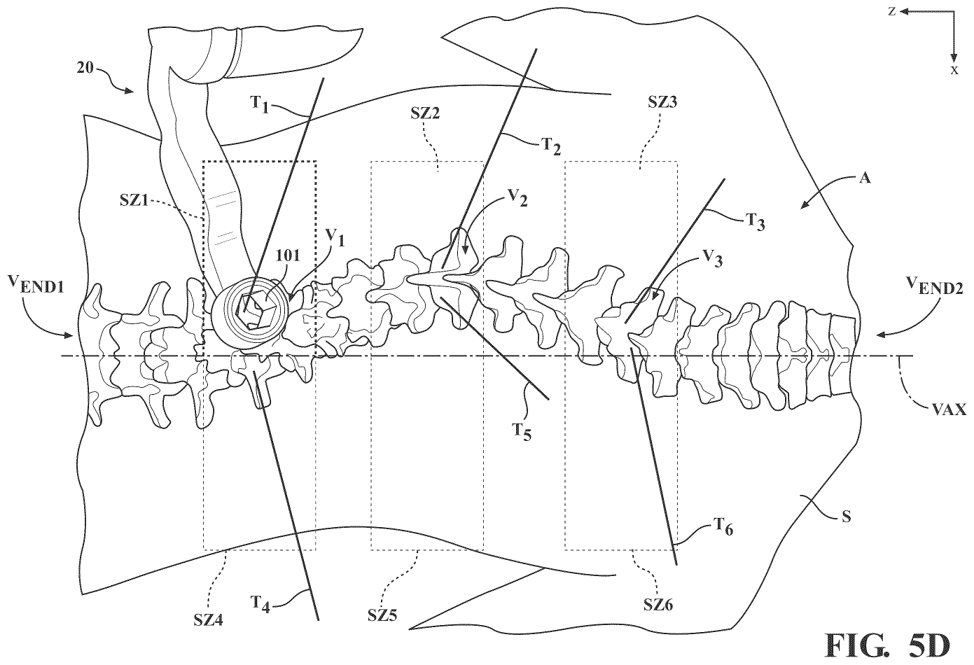

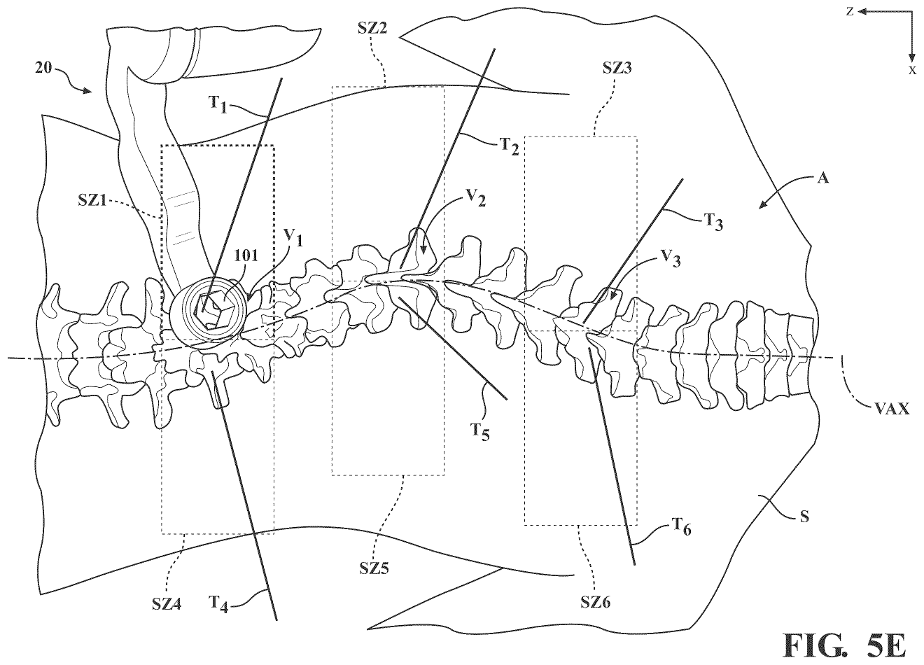

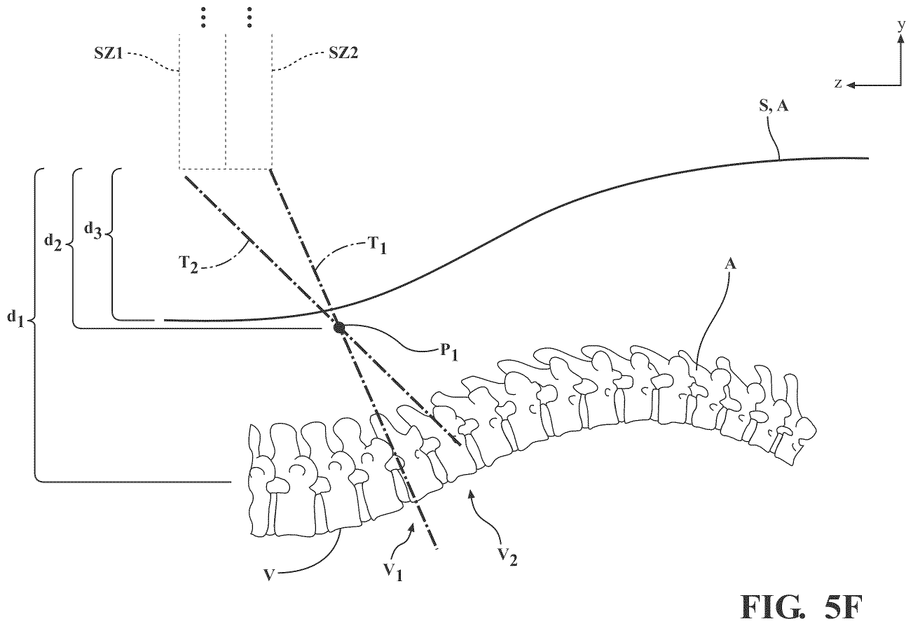

Herein, some steps of the method 200 are relative to FIGS. 5A-5E. In FIGS. 5A-5F, a patient 12 is placed in a prone position, with a head of the patient located toward a right side of the Figure and feet of the patient 12 located toward a left side of the Figure. In the instances of FIGS. 5A-5F, a vertebra V of the patient 12 is shown as the anatomy A of the patient 12, with the vertebra V including a plurality of vertebral bodies. For instance, vertebral bodies V1-V5 are indicated in FIG. 5A. Additionally, the vertebra V includes a vertebral axis VAX. The vertebral axis VAX may be defined in many ways. In one example, the vertebral axis VAX is a line or contour that follows a center of one or more vertebral bodies. For instance, the vertebral axis VAX may be defined as a straight line between two spaced apart vertebral bodies. Such an instance is shown in FIG. 5D, where the vertebral axis VAX is defined as a straight line between two vertebral bodies V_{END1} and V_{END2}. In another instance, such as the instance of FIG. 5E, the vertebral axis VAX may be an average centerline among a plurality of vertebral bodies. The vertebral axis VAX may be linear, curved, or curvilinear.

a. Tracking a Pose of the End Effector and a Pose of the Anatomy

The navigation system 32 may be configured to perform steps 202 and 204 of the method 200. For example, in instances where the tool tracker 106 (see FIG. 1) is coupled to the end effector 20, the localizer 44 of the navigation system 32 may be configured to track a pose of the end effector 20 by tracking the tool tracker 106. In instances where one or more patient trackers, such as the first and/or second patient trackers 54, 56 (see FIG. 1), are affixed to the patient 12, the localizer 44 may track the patient trackers 54, 56 to track a pose of an anatomy A of the patient 12. For example, in the instance of FIG. 1, the first patient tracker 54 is affixed to a vertebra V of the patient 12. The localizer 44 may track the first patient tracker 54 to determine a pose of the vertebra V of the patient 12.

The method 200 may include steps of tracking the pose of end effector 20 and the pose of the anatomy A in the localizer coordinate system LCLZ (see FIG. 1). Additionally, the method 200 may include steps of transforming the pose of end effector 20 and the pose of the anatomy A from the localizer coordinate system LCLZ to the manipulator coordinate system MNPL using known transformation/registration techniques. For instance, the localizer 44 may determine coordinates of the tool tracker 106 in the localizer coordinate system LCLZ to track a pose of the end effector 20 in the localizer coordinate system LCLZ, and the localizer 44 may determine coordinates of a patient tracker in the localizer coordinate system LCLZ to track a pose of the anatomy A of the patient 12 in the localizer coordinate system LCLZ. The localizer 44 may then transform the coordinates of the tool tracker 106 and the coordinates of a patient tracker from the localizer coordinate system LCLZ to the manipulator coordinate system MNPL. In this way, the manipulator 14 may operate the robotic arm 18A based on the tracked pose of end effector 20 and based on the tracked pose of the anatomy A by referencing the coordinates of the end effector 20 and the coordinates of the anatomy A in the manipulator coordinate system MNPL. For instance, the controller 30 may generate a virtual boundary based on the pose of the anatomy A and the manipulator 14 may operate the robotic arm 18A such that the end effector 20 avoids the virtual boundary.

In some instances, the controller 30 may be configured to perform step 202 of tracking a pose of the end effector 20. In such an instance, the controller 30 may track a pose of the end effector 20 based on kinematic data of the manipulator 14. For example, in the instance of FIG. 1, the end effector 20 is shown as a distal flange of the robotic arm 18A. In such instances, the method may include a step of tracking the pose of the end effector 20 in the manipulator coordinate system MNPL. For instance, the controller 30 may determine coordinates of the end effector 20 in the manipulator coordinate system MNPL to track a pose of the end effector 20 in the manipulator coordinate system MNPL.

The method 200 may be configured to perform the steps described herein based on the pose of the end effector 20 and/or the pose of the anatomy A. For example, the controller 30 may associate the target trajectory T with the anatomy A during step 206 based on the pose of the anatomy A. As another example, the controller 30 may determine that the end effector 20 is within the trajectory selection zone SZ during step 212 of selecting a target trajectory T. As another example, the controller 30 may determine that the end effector 20 is within a prevention zone (described in greater detail below) based on the pose of the end effector 20. As another example, a display may provide an indication that the end effector 20 is closer to one of the target trajectories T and/or one of the trajectory selection zones SZ based on the pose of the end effector 20. As another example, the controller 30 may determine a position of the end effector 20 along the target trajectory T during step 216 of constrain movement of the end effector 20 to the target trajectory T.

b. Associating a Target Trajectory

The controller 30 may be configured to perform the step 206 of associating a target trajectory T with the anatomy A of the patient 12. Specifically, during step 206, the boundary generator 66 of the controller 30 may be configured to define the target trajectory T and associate the target trajectory T with the anatomy A. In instances where the anatomy A is a vertebra V, the target trajectory T may indicate an ideal trajectory for drilling into and/or inserting a pedicle screw into the vertebra V. The end effector 20 may be aligned with the target trajectory T to drill into and/or insert a pedicle screw into the vertebra V along the target trajectory T.

The controller 30 may associate more than one target trajectory T to the anatomy A. For example, in the instance of FIG. 5A, the anatomy A of the patient 12 is the vertebra V and the target trajectories T₁-T₁₀ are associated with the vertebra V. Additionally, in instance where the anatomy A is a vertebra V, the controller 30 may associate more a target trajectory with a vertebral body of the vertebra V. For example, in the instance of FIG. 5A, the target trajectory T₁ is associated with the vertebral body V₁, the target trajectory T₂ is associated with the vertebral body V₂, the target trajectory T₃ is associated with the vertebral body V₃, the target trajectory T₄ is associated with the vertebral body V₄, and the target trajectory T₅ is associated with the vertebral body V₅. Furthermore, in instances where the anatomy A is a vertebra V including vertebral bodies, the controller 30 may be configured to associate more than one target trajectory T with a vertebral body of the vertebra V. For example, the controller 30 may associate a target trajectory T with a right side of a vertebral body and a target trajectory T with a left side of the vertebral body. For example, in the instance of FIG. 5A, the target trajectories T₁, T₆ are associated with a left and right side of vertebral body V₁, respectively; the target trajectories T₂, T₇ are associated with a left and right side of the vertebral body V₂, respectively; the target trajectories T₃, T₈ are associated with a left and right side of the vertebral body V₃, respectively; the target trajectories T₄, T₉ are associated with a left and right side of the vertebral body V₄, respectively; and the target trajectories T₅, T₁₀ are associated with a left and right side of the vertebral body V₅, respectively.

The controller 30 may be configured to associate a target trajectory T with the anatomy A according to many methods/sources. In one example, the target trajectory T may be based on a planned pedicle screw. For example, pre-and intra-operative imaging data of the patient 12 may be acquired and a virtual pedicle screw may be positioned and/or oriented relative to the anatomy A, such as a vertebral body. For instance, the target trajectory T may be a virtual axis of the virtual pedicle screw. In another example, the trajectory may be defined without planning a screw. For example, the target trajectory T may be a pedicle entry trajectory planned relative to the anatomy A in the medical imaging data. In another example, the target trajectory T may be defined based on surgeon preference. The target trajectory T may be defined on-the-fly by a user using a tracked surgical device such as a probe, a cutting tool, or the surgical system 10. In another instance, the target trajectory T may be automatically generated or manually defined. When automatically generated, a target trajectory T to be associated with a vertebral body may be based on an analysis of a population of vertebral bodies similar to the target vertebral body.

c. Defining a Trajectory Selection Zone

The controller 30 may be configured to define the trajectory selection zone SZ during step 208. Specifically, the boundary generator 66 of the controller 30 may be configured to define the trajectory selection zone SZ and associate the trajectory selection zone SZ with a target trajectory T during step 208. As will be described in greater detail below, the trajectory selection zones SZ allow a target trajectory T to be selected for alignment by the end effector 20.

Example trajectory selection zones SZ are shown in FIG. 5A. The controller 30 may be configured to associate a trajectory selection zone SZ with each target trajectory T associated with the anatomy A. For example, in the instance of FIG. 5A, the anatomy A of the patient 12 is the vertebra V and the target trajectories T₁-T₁₀ are associated with the vertebra V. Correspondingly, the controller 30 associates each of the trajectory selection zones SZ1-SZ10 with each of the target trajectories T₁-T₁₀, respectively. As will be described in greater detail below, a target trajectory T may be selected for alignment by the end effector 20 by moving the end effector 20 into the corresponding trajectory selection zone SZ. For example, as shown in FIG. 5A, the end effector 20 includes a guide tube 101 and the guide tube 101 of the end effector 20 is moved into the trajectory selection zone SZ2 and the controller 30 automatically selects the target trajectory T₂.

In some instances, the controller 30 may be configured to associate a trajectory selection zone SZ to a target trajectory T based on whether the target trajectory T is a left-side or a right-side target trajectory T. Referring to FIG. 5A, the target trajectories T₁, T₂, T₃, T₄, T₅ are left side target trajectories and the target trajectories T₆, T₇, T₈, T₉, T₁₀ are right-side target trajectories. The controller 30 then associates a left-side trajectory selection zone SZ1, SZ2, SZ3, SZ4, SZ5 with each of the left-side target trajectories T₁, T₂, T₃, T₄, T₅ and a right-side trajectory selection zone SZ6, SZ7, SZ8, SZ9, SZ10 with each of the right-side target trajectories T₆, T₇, T₈, T₉, T₁₀, where a left-side trajectory selection zone is defined as being located at a left-side of the vertebral axis VAX, as indicated in FIG. 5A, and where a right-side trajectory selection zone is defined as being is located at a right-side of the vertebral axis VAX, as indicated in FIG. 5A.

Additionally, the controller 30 may identify whether a target trajectory T is a left-side target trajectory or a right-side target trajectory. For example, the controller 30 may identify whether a target trajectory T is a left-side target trajectory or a right-side target trajectory based on a position and/or orientation of the target trajectory T. For instance, the controller 30 may identify whether a target trajectory T is a left-side target trajectory or a right-side target trajectory based on a position of a vertebra-adjacent end of a target trajectory T relative to the vertebral axis VAX, the vertebra-adjacent end of a target trajectory T being defined as the end of the target trajectory T that is adjacent to the vertebra V. As another example, the controller 30 may identify whether a target trajectory T is a left-side target trajectory or a right-side target trajectory based on a number of target trajectories T associated with a vertebral body. Furthermore, the controller 30 may identify whether a target trajectory T is a left-side target trajectory or a right-side target trajectory based on a user input. For instance, an operator may indicate whether a target trajectory T is a left-side or a right-side target trajectory during the step 206 of associating the target trajectory T with the anatomy A.

In some instances, the controller 30 may be unable to identify whether a target trajectory T is a left-side target trajectory or a right-side target trajectory. For example, the controller 30 may be unable to identify whether a target trajectory T is a left-side target trajectory or a right-side target trajectory due to a position and/or orientation of the target trajectory T. For instance, portions of the target trajectory T may be located on either side of the target trajectory T. Such a phenomenon may occur as a result of irregularities in the curvature of the vertebra V. As another example, the controller 30 may be unable to identify whether a target trajectory T is a left-side target trajectory or a right-side target trajectory as a result of insufficient data at the time of surgical planning. For instance, an operator may not have indicated whether a target trajectory T is a left-side or a right-side target trajectory during the step 206 of associating the target trajectory T with the anatomy A.

In instances where the controller 30 is unable to identify whether a target trajectory T is a left-side target trajectory or a right-side target trajectory, the controller 30 may associate a left-side trajectory selection zone and a right-side trajectory selection zone with the target trajectory T. For example, FIG. 5B provides an exemplary instance where the controller 30 is unable to determine whether the target trajectories T₁, T₇ are left or right-side target trajectories. As shown, the controller 30 may associate both a left-side and right-side trajectory selection zone SZ1, SZ6 with the target trajectory T₁ and both a left-side and right-side trajectory selection zone SZ2, SZ7 with the target trajectory T₇. In such instances, target trajectory T₁ may be automatically selected after the end effector 20 is moved into either trajectory selection zone SZ1, SZ6 and target trajectory T₇ may be automatically selected after the end effector 20 is moved into either trajectory selection zone SZ2, SZ7.

The trajectory selection zone SZ may be defined as including any suitable shape and/or dimensions during step 208.

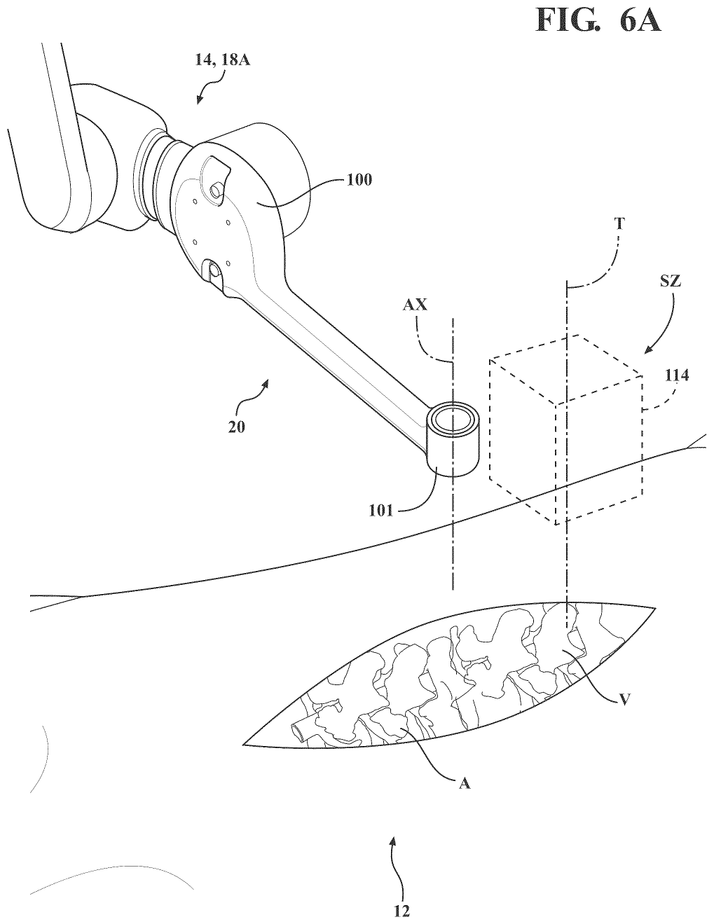

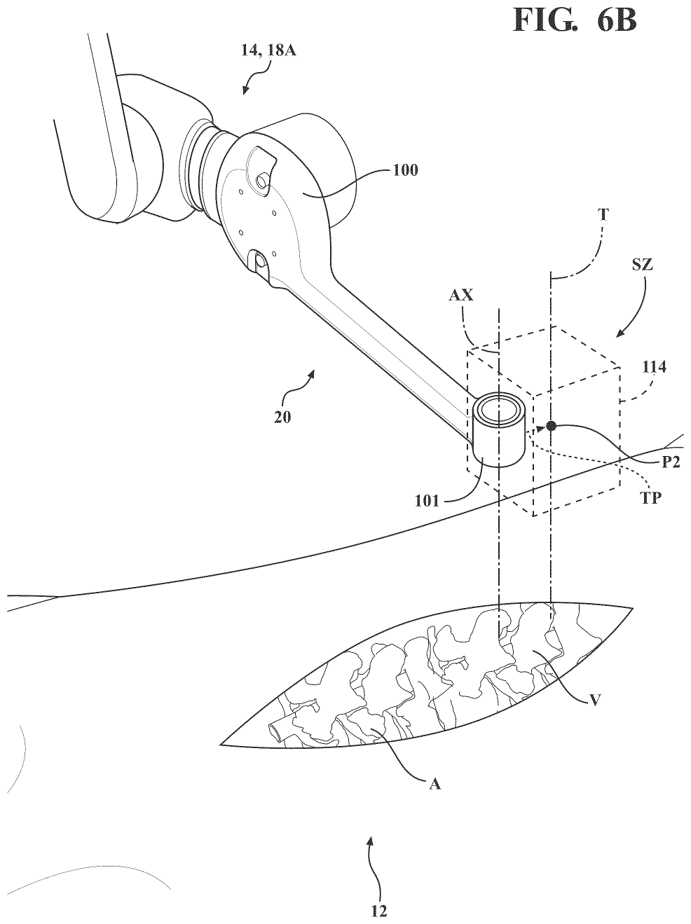

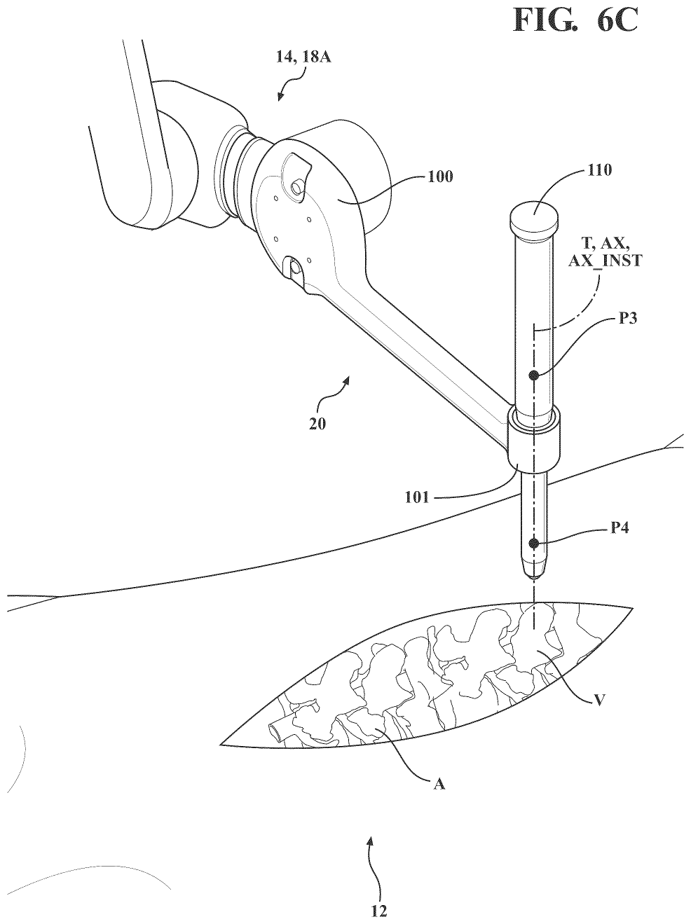

The trajectory selection zone SZ may be defined as including any three-dimensional shape and/or including any cross-sectional shape during step 208. For example, the trajectory selection zones SZ are shown in FIG. 5A as including a rectangular cross-section. Additionally, referring to FIGS. 6A-6C, the trajectory selection zones SZ are shown as including a rectangular prism shape. In alternative instances, the trajectory selection zone SZ may include any other suitable shape. For example, the trajectory selection zone SZ may include a spherical, ellipsoidal, conical, cylindrical shape, or the like. As another example, the trajectory selection zone SZ may include any polygonal cross-sectional shape.

The trajectory selection zones SZ may be defined as including any suitable dimensions during step 208. For example, the trajectory selection zones SZ may be defined such that a height, width, and/or length for each trajectory selection zone SZ is equivalent. As yet another example, the trajectory selection zones SZ may be defined such that a height, width, and/or length of each trajectory selection zone SZ does not exceed a predetermined maximum value and/or is not below a predetermined minimum value. As another example, the trajectory selection zones SZ may be defined such that a height, width, and/or length of the trajectory selection zones SZ provides suitably sized volumes for the end effector 20 to enter during selection of the target trajectory T. In one such instance, the trajectory selection zones SZ may be defined such that a height, width, and/or length of the trajectory selection zones SZ provide suitably sized volumes such that the entire guide tube 101 may be disposed within the trajectory selection zones SZ.

In some instances, the controller 30 may define a size of the trajectory selection zones SZ such that a height, width, and/or length of each trajectory selection zone SZ using a fixed value. For example, referring to FIG. 5A, the controller 30 may define a height hₓ along the x-axis of the trajectory selection zones SZ1-SZ10 using a fixed value. As another example, referring to FIG. 5C, the controller 30 may define a height h_{y} along the y-axis of the trajectory selection zones SZ1-SZ10 using a fixed value.

In some instances, the controller 30 may calculate a height, width, and/or length of each trajectory selection zone SZ. In one such instance, the controller 30 may group the target trajectories T and calculate dimensions of the associated trajectory selection zones SZ based on the grouping. For example, referring to FIG. 5A, the controller 30 groups the left-side target trajectories and the right-side target trajectories. As shown, the left-side target trajectories T₁, T₂ are grouped into a first grouping G1, the left-side target trajectories T₃, T₄, T₅ are grouped into a second grouping G2, the right-side target trajectories T₆, T₇ are grouped into a third grouping G3, and the right-side target trajectories T₈, T₉, T₁₀ are grouped into a fourth grouping G4. The controller 30 may group the target trajectories T based on a distance between adjacent target trajectories T. For example, the controller 30 may group adjacent target trajectories T based on determining whether a distance between the adjacent target trajectories T is below a threshold value. As shown in FIG. 5A, each trajectory selection zone SZ associated with target trajectories T of a single grouping includes the same width along the z-axis. For example, trajectory selection zones SZ1, SZ2 include a first width w_{1z} along the z-axis, trajectory selection zones SZ3, SZ4, SZ5 include a second width w_{2z} along the z-axis, trajectory selection zones SZ6, SZ6 include a third width w_{3z} along the z-axis, and trajectory selection zones SZ8, SZ9, SZ10 include a fourth width w_{4z} along the z-axis.

The controller 30 may calculate the widths w_{1z}, w_{2z}, w_{3z}, w_{4z} such that a portion of each target trajectory T₁-T₁₀ (e.g. a vertebra-adjacent end of each target trajectory T₁-T₁₀) is within the associated trajectory selection zone SZ1-10 and such that the trajectory selection zones SZ1-SZ10 do not overlap. Additionally, when calculating the dimensions of the trajectory selection zones SZ, the controller 30 may calculate the dimensions such that a portion of each target trajectory T₁-T₁₀ (e.g. a vertebra-adjacent end of each target trajectory T₁-T₁₀) is within the associated trajectory selection zone SZ1-10, such that the trajectory selection zones SZ1-SZ10 do not overlap, and such that the calculated dimensions do not exceed a predetermined maximum value. For instance, referring to FIG. 5B, the fourth grouping G4 includes only the target trajectory T₉. As such, the controller 30 calculates the width w_{4z} as being the maximum predetermined value.

The trajectory selection zone SZ may be defined as including any suitable location during step 208.

The trajectory selection zones SZ may be positioned such that the trajectory selection zones SZ are non-overlapping to facilitate selection of the associated target trajectory T during step 212. As shown in FIG. 5A, the trajectory selection zones SZ1-SZ10 are defined such that the trajectory selection zones SZ1-SZ10 are non-overlapping. In this way, the end effector 20 may be moved to be within a single trajectory selection zone SZ at a time, ensuring that a single target trajectory will be selected during step 212.

The trajectory selection zones SZ may also be positioned relative to the anatomy A to facilitate selection of the associated target trajectory T during step 212.

For example, the trajectory selection zones SZ may be positioned relative to the vertebral axis VAX. Referring to FIG. 5D, the trajectory selection zones SZ1-SZ6 may be positioned along the vertebral axis VAX on the z-x plane. In instances where the vertebral axis VAX is curved, the trajectory selection zones SZ may also be positioned along the vertebral axis VAX. Such an instance is shown in FIG. 5E, where the vertebral axis VAX follows a curvature of the vertebra V. As shown, the trajectory selection zones SZ1-SZ6 are positioned along the vertebral axis VAX on the z-x plane.

As another example, the trajectory selection zones SZ1-SZ6 may be positioned based on the grouping of the target trajectories T. For example, referring to FIG. 5C, the trajectory selection zones SZ1-SZ10 are positioned along the z-y plane based on an average location along the y-axis of the grouped target trajectories T₁-T₁₀. For instance, trajectory selection zones SZ1-SZ2 are positioned along the z-y plane based on an average location along the y-axis of target trajectories T₁, T₂. Similarly, trajectory selection zones SZ3-SZ5 are positioned along the z-y plane based on an average location along the y-axis of target trajectories T₃, T₄, T₅. trajectory selection zones SZ6-SZ6 are positioned along the z-y plane based on an average location along the y-axis of target trajectories T₆, T₇, and the trajectory selection zones SZ8-SZ10 are positioned along the z-y plane based on an average location along the y-axis of target trajectories T₈, T₉, T₁₀. In such instances a location along the y-axis of a target trajectory T may be determined using any suitable method and/or means. For example, the location along the y-axis of a target trajectory T may correspond to a y-coordinate of the vertebra-adjacent end of the target trajectory T. As another example, the location along the y-axis of a target trajectory T may correspond to a y-coordinate of any point along the target trajectory T that intersects with the vertebra V.

As yet another example, the trajectory selection zones SZ may be located at a predetermined distance from the anatomy A to allow a target trajectory T to be selected without being directly adjacent to the anatomy A. Referring to FIG. 5F, the trajectory selection zones SZ1, SZ2 may be located at a predetermined distance d₁ from the vertebra V. As another example, the trajectory selection zones SZ1, SZ2 may be located at a predetermined distance d₃ above a skin surface S of the patient 12.

Additionally, the trajectory selection zones SZ may be positioned relative to an intersection of target trajectories T to facilitate selection of the associated target trajectory T during step 212. As will be explained in greater detail below, during step 210, the controller 30 may operate the robotic arm 18A in the free mode FM, allowing an operator to direct movement of the end effector 20 to enter a trajectory selection zone SZ and allowing the controller 30 to automatically select a target trajectory T. However, in instances where more than one target trajectory T is associated with the anatomy A, an operator may have difficulty selecting a specific target trajectory T during step 212. For example, in some instances, multiple target trajectories T may be defined and associated with the anatomy A, with the multiple target trajectories T extending in different directions. In such instances, the target trajectories T may intersect or converge, and an operator may have difficulty selecting a specific target trajectory T during step 212. As such, by providing the trajectory selection zones SZ above the point of convergence, the trajectory selection zones SZ are located at a distance where the converging target trajectories T would be diverging from one another. For instance, referring to FIG. 5F, two-dimensional projections of the first and second target trajectories T₁, T₂ along the cross-sectional plane of FIG. 5F are shown. Additionally, the two-dimensional projections of the first and second target trajectories T₁, T₂ are shown intersecting at a point P₁. As follows, the trajectory selection zones SZ1, SZ2 may be located at a predetermined distance d₂ from the point P₁ to facilitate selection of either the first target trajectory T₁ or the second target trajectory T₂ during step 212.

It is contemplated that the trajectory selection zones SZ may be positioned using any suitable method and/or means. For example, the trajectory selection zones SZ may be positioned such that the trajectory selection zones SZ are offset from the vertebral axis VAX. As another example, the trajectory selection zones SZ may be positioned such that the trajectory selection zones SZ are spaced from one another. As another example, an operator may adjust a position of the trajectory selection zones SZ using an input device, such as one or more of the input devices 40, 42, 43.

relative to the anatomy A to facilitate selection of the associated target trajectory T during step 212.

d. Operation in the Free Mode

During step 210, the controller 30 may operate the robotic arm 18A in the free mode FM. FIG. 6A illustrates an example operation of the robotic arm 18A in the free mode FM during step 210. In the instance of FIG. 6A, the end effector 20 includes a guide tube 101. As shown, the guide tube 101 is not within the trajectory selection zone SZ and the guide tube 101 is not aligned with the target trajectory T. In the free mode FM, the robotic arm 18A is freely moveable such that the guide tube 101 is also freely moveable. During operation of the robotic arm 18A in the free mode FM, an operator may manually direct movement of the end effector 20 to move the guide tube 101 toward the trajectory selection zone SZ.

During step 212, the controller 30 may automatically select a target trajectory T in response to determining that the end effector 20 is within the associated trajectory selection zone SZ. Step 212 occurs in response to movement of the robotic arm 18A in the free mode FM during step 210. As such, during operation in the free mode FM, an operator may direct movement of the end effector 20 to move the end effector 20 to be within a trajectory selection zone SZ to trigger selection of the associated target trajectory T.

The controller 30 may determine that the end effector 20 is within the trajectory selection zone SZ using any suitable method. For example, in instances where the end effector 20 includes the guide tube 101, such as the instance of FIG. 6B, the controller 30 may determine that the end effector 20 is within the trajectory selection zone SZ by determining that the guide tube 101 of the end effector 20 is within the trajectory selection zone SZ. The controller 30 may determine that the end effector 20 is within the trajectory selection zone SZ by determining that the guide tube 101 is with a perimeter 114 of the trajectory selection zone SZ. Additionally, or alternatively, the controller 30 may determine that a portion of the guide tube 101 is within the trajectory selection zone SZ by determining that a majority of the guide tube 101 is within the trajectory selection zone SZ. For example, in instance of FIG. 5A, the controller 30 may determine that the end effector 20 is within the trajectory selection zone SZ2 as a majority of the guide tube 101 is located within the trajectory selection zone SZ2, even though a portion of the guide tube 101 is located within the trajectory selection zone SZ1.

Generally, the trajectory selection zones SZ provide an operator-friendly means of selecting a target trajectory T during step 212. As previously discussed, the trajectory selection zones SZ may be shaped, sized, and positioned to facilitate selection during step 212.

e. Operation in the Automatic Mode

During step 214, the controller 30 may operate the robotic arm 18A in the automatic mode, whereby the robotic arm 18A is automatically moved to align the end effector 20 with the target trajectory T. Step 214 occurs in response to a selection of a target trajectory T during step 212. In other words, once the controller 30 determines that the end effector 20 is within the trajectory selection zone SZ and selects the associated target trajectory T during step 212, the controller 30 may control the robotic arm 18A in the automatic mode AM to align the end effector 20 to the selected target trajectory T.

FIG. 6B illustrates an example operation of the robotic arm 18A in the automatic mode AM during step 214. As shown, the controller 30 operates the robotic arm 18A in the automatic mode AM upon determining that the guide tube 101 is within the trajectory selection zone SZ and selecting the target trajectory T. Also shown, during operation of the robotic arm 18A in the automatic mode AM, the end effector 20 is moved along a tool path TP to align the guide tube 101 with the selected target trajectory T. In some instances, a length of the tool path TP may be minimized to minimize movement of the end effector 20 during alignment of the end effector 20. In such instances, the tool path TP may be based on a point along the selected target trajectory T closest to the position of the end effector 20. In the instance of FIG. 6B, a point P₂ along the target trajectory T may be determined by the controller 30 as being a point along the selected target trajectory T to which the guide tube 101 is closest. The tool path generator 68 may then generate the tool path TP based on the location of the guide tube 101 and the point P₂ and the end effector 20 may be moved along the tool path TP in the automatic mode AM to align the end effector 20 with the selected target trajectory T.

The trajectory selection zones SZ may be positioned to reduce a length of a tool path TP along which the end effector 20 is moved during step 214. For example, referring to FIG. 5A, the trajectory selection zones SZ1-SZ10 are positioned proximate to the associated target trajectories T1-T10. Advantageously, by positioning the trajectory selection zones SZ proximate to the associated target trajectories T, the end effector 20 is located proximate to the selected target trajectory T after the end effector 20 is determined to be within the trajectory selection zone SZ. In this way, the length of the tool path TP along which the end effector 20 is moved during step 214 is reduced.

Additionally, the trajectory selection zones SZ may be positioned based on the anatomy A of the patient 12 to reduce a length of a tool path TP along which the end effector 20 is moved during step 214. For example, in the instance of FIG. 5C, the vertebra V includes a curvature, causing displacement of the vertebral bodies V₁, V₂, V₃. Displacement of the vertebral bodies V₁, V₂, V₃ affects a location of the target trajectories T, as the target trajectories T are associated with the vertebra V. In such instances the trajectory selection zones SZ1, SZ3, SZ4, SZ6 may be positioned based on a location of the vertebral bodies V₁, V₃, without being positioned about an axis VAX of the vertebra V, such that the trajectory selection zones SZ1, SZ3, SZ4, SZ6 are proximate to the target trajectories T₁-T₆, despite curvature of the vertebra V. In this way, the end effector 20 is located proximate to the selected target trajectory T after the end effector 20 is determined to be within the trajectory selection zone SZ, reducing the length of the tool path TP along which the end effector 20 is moved during step 214.

The method 200 may include a step of associating a prevention zone with a target trajectory T to ensure that a length of a tool path TP along which the end effector 20 is moved during step 214 does not exceed a threshold length. An example prevention zone 112 is shown in FIG. 4. The prevention zone 112 may be associated with a target trajectory T by the boundary generator 66 of the controller 30. Referring to FIG. 5A, the controller 30 associates a prevention zone 112 (i.e., prevention zone 112-1, prevention zone 112-2, etc.) to each of the target trajectories T₁-T₁₀. In instances where a prevention zone 112 is associated with a target trajectory T, the controller 30 may be configured to automatically select the target trajectory T during step 212 in response to determining that the end effector 20 is within both the trajectory selection zone SZ and the prevention zone 112. In this way, the prevention zone 112 provides an additional safeguarding measure to ensure that the end effector 20 does not move a substantial distance while being automatically aligned with the selected target trajectory T during step 214.

The prevention zones 112 may include any suitable shape. For example, the prevention zone 112 may include any suitable three-dimensional shape and/or any suitable two-dimensional cross-sectional shape. In the instance of FIGS. 4 and 5A, the prevention zones 112 include a spherical shape and are shown as including a circular cross-sectional shape. In other instances, the prevention zones 112 may include a rectangular, ellipsoidal, conical, cylindrical shape, or the like. As another example, the trajectory selection zone SZ may include any polygonal cross-sectional shape.

The prevention zones 112 may include any suitable size and/or position for ensuring that a length of a tool path TP along which the end effector 20 is moved during step 214 does not exceed a threshold length. For example, the prevention zone 112 may include a spherical shape, wherein a radius of the spherical shape is based on a maximum allowable distance the end effector 20 may travel during automatic alignment. In other instances, a prevention zone 112 may be sized such that the prevention zone 112, which is associated with a target trajectory T, surrounds the trajectory selection zone SZ associated with the same target trajectory T.

f. Operation in the Haptic Mode

During step 216, the controller 30 may operate the robotic arm 18A in the haptic mode, whereby movement of the end effector 20 is constrained to the selected target trajectory T. The controller 30 constrains the end effector 20 to the selected target trajectory T during step 216 in response to the end effector 20 being aligned with the selected target trajectory T during step 214. In the instance of FIG. 6C, the guide tube 101 is aligned with the selected target trajectory T, as illustrated by the alignment of the axis AX of the guide tube 101 to the target trajectory T. Additionally, the guide tube 101 is constrained to the selected target trajectory T to maintain alignment of the axis AX to the selected target trajectory T.

During operation in the haptic mode HM, the end effector 20 is constrained to the selected target trajectory T such that the end effector 20 may move along the selected target trajectory T. For example, the guide tube 101 of FIG. 6C may move in a direction that maintains alignment of the axis AX of the guide tube 101 to the selected target trajectory T, while being constrained from moving in a direction that would cause misalignment of the axis AX to the selected target trajectory T.

In some instances, the controller 30 may define one or more points along the selected target trajectory T and the end effector 20 may be constrained to the selected target trajectory T based on a position of the one or more points. In one such instance, the controller 30 may define a point along the selected target trajectory T and constrain movement of the end effector 20 such that the end effector 20 may move along the selected target trajectory without passing the point. In another instance, the controller 30 may define more than one point along the selected target trajectory T and constrain movement of the end effector 20 such that the end effector 20 may move along the selected target trajectory between the points. In the instance of FIG. 6C, the controller 30 defines a first point P₃ and a second point P₄ along the target trajectory T. In such an instance, the controller 30 may then constrain the end effector 20 such that the end effector 20 may move along the selected target trajectory T above the point P₄. In another instance, the controller 30 may constrain the end effector 20 such that the end effector 20 may move along the selected target trajectory T below the point P₃. In yet another instance, the controller 30 may constrain the end effector 20 such that the end effector 20 may move along the selected target trajectory T between the points P₃, P₄.

In some instances, the controller 30 may cease operation in the haptic mode HM based on movement of the end effector 20 along the selected target trajectory T. For example, the controller 30 may cease operation in the haptic mode HM in response to movement of the end effector 20 along the selected target trajectory T above the point P₃. In some instances, the controller 30 may cease operation in the haptic mode HM in response to movement of the end effector 20 along the selected target trajectory T below the point P₄. In some instances, the controller 30 may cease operation in the haptic mode HM in response to movement of the end effector 20 along the selected target trajectory T above the point P₃ or below the point P₄. In such instances, the controller 30 may cease operation in the haptic mode HM and operate the robotic arm 18A in any suitable operating mode. For example, the controller 30 may cease operation in the haptic mode HM and operate the robotic arm 18A in the free mode FM. As another example, the controller 30 may cease operation in the haptic mode HM and operate the robotic arm 18A in the automatic mode AM and automatically move the end effector 20. For instance, the controller 30 may cease operation in the haptic mode HM in response to movement of the end effector 20 along the selected target trajectory T above the point P₃ and operate the robotic arm 18A in the automatic mode AM to automatically align the end effector 20 to the selected target trajectory T and to position the end effector 20 below the point P₁.

The points P₃, P₄ may be defined using any suitable means. For example, referring to FIG. 6C, the first point P₃ may be located at a first position along the target trajectory T and the second point P₄ may be located at a second position along the target trajectory T, wherein the first position is further from the anatomy A than the second position. As another example, the points P₃, P₄ may be defined based on the tracked pose of the anatomy A. For instance, the point P₃ along the target trajectory T may be defined based on a first predetermined distance from the anatomy A and the point P₄ may be defined based on a second predetermined distance from the anatomy. Additionally, the points P₃, P₄ may be defined prior to, after, or during any suitable step of the method 200. For example, the points P₃, P₄ may be defined in response to the end effector 20 being automatically aligned with the target trajectory T during step 214. As another example, the points may be defined when the target trajectory T is associated with the anatomy A during step 206.

As shown in FIG. 6C, the robotic arm 18A may support a surgical instrument 110. In the instance of FIG. 6C, the surgical instrument 110 is temporarily affixed to the guide tube 101 of the end effector 20 such that the guide tube 101 and the robotic arm 18A support the surgical instrument 110. In other instances, the end effector 20 may include the surgical instrument 110, with the surgical instrument 110 being directly coupled to the robotic arm 18A such that the robotic arm 18A supports the surgical instrument 110. The surgical instrument 110 may be supported by the robotic arm 18A during any step of the method 200. For example, the surgical instrument 110 may be supported by the robotic arm 18A prior to or after the step 216 of operating in the haptic mode HM. The surgical instrument 110 may be supported by the robotic arm 18A during the step 210 of operating in the free mode FM or during the step 214 of operating in the automatic mode AM.

In instances where the robotic arm 18A supports a surgical instrument 110, the controller 30 may align the end effector 20 during step 214 such that the surgical instrument 110 is also aligned with the selected target trajectory T. For example, referring to FIG. 6C, a portion of the surgical instrument 110 may extend along an axis AX_INST. During step 214, the controller 30 may align the end effector 20 such that the axis AX_INST of the surgical instrument 110 is also aligned with the selected target trajectory T. During step 216, the controller 30 may operate the robotic arm 18A in the haptic mode HM to maintain alignment of the surgical instrument 110 to the selected target trajectory T by maintaining alignment of the axis AX_INST and the selected target trajectory T.

g. Input Devices

The method 200 and any method described herein may include a step of detecting an input from the one or more input devices 40, 42, 43. In such instances, any one or more of the steps 202-216 of the method 200 may occur in response to the step of detecting an input from the one or more input devices 40, 42, 43. For example, the step 214 of automatically aligning the end effector 20 may occur in response to detecting an input from the one or more input devices 40, 42, 43. In one such instance, an operator may provide an input to one or more input devices 40, 42, 43 (e.g. pressing the user-actuatable foot pedal of the footswitch 43) to initiate step 214 and continue providing the input (e.g. holding the user-actuatable foot pedal) throughout the duration of step 214 as the controller 30 automatically aligns the end effector 20 to a selected target trajectory T. In some instances, an operator may be notified (e.g. via display 38) that a target trajectory T has been selected by the controller 30 during step 212. In such instances, the operator may provide an input to one or more input devices 40, 42, 43 (e.g. pressing the user-actuatable foot pedal of the footswitch 43) to confirm selection of the target trajectory T and initiate step 214 of automatically aligning the end effector 20. As another example, the step 216 of operating the robotic arm 18A in the haptic mode HM may occur in response to detecting an input from the one or more input devices 40, 42, 43. In one such instance, an operator may release the user-actuatable foot pedal of the footswitch 43 after the end effector 20 is aligned with the selected target trajectory T to trigger step 216 and initiate operation in the haptic mode HM.

The method 200 and any method described herein may include a step of implementing haptic and/or audible feedback to a component of the surgical system 10. The haptic and/or audible feedback may be implemented by the controller 30 and may be implemented to any suitable device of the surgical system 10. For example, the method 200 may include a step of implementing haptic feedback to the end effector 20 and/or a step of implementing haptic and/or audible feedback to one or more of the input devices 40, 42, 43. In one such instance, the method 200 may include a step of implementing haptic feedback to the footswitch 43 by providing a vibratory force.

The method 200 may include a step of implementing haptic and/or audible feedback prior to, during, or after any of the steps 202-216 of the method 200 to provide an indication and/or notification to an operator.

For example, the method 200 may include a step of implementing haptic and/or audible feedback in response to a target trajectory T being selected during step 212. For example, in instances where the operator presses the user-actuatable foot pedal of the footswitch 43 to initiate step 214 of operating in the automatic mode AM, such haptic and/or audible feedback may indicate that the end effector 20 is within a trajectory selection zone SZ and that the operator may press the foot pedal to automatically align the end effector 20 with the associated target trajectory T. In one such instance, the method 200 may include a step of implementing haptic and/or audible feedback in response to the end effector 20 entering the perimeter 114 of the trajectory selection zone SZ. In another instance, the method 200 may include a step of implementing haptic and/or audible feedback in response to a majority of the end effector 20 being within the trajectory selection zone SZ.

As another example, the method 200 may include a step of implementing haptic and/or audible feedback in response to the end effector 20 being aligned with a selected target trajectory T during step 214. In instances where an operator holds the user-actuatable foot pedal throughout the duration of step 214 as the controller 30 automatically aligns the end effector 20 with the selected target trajectory T and releases the user-actuatable foot pedal of the footswitch 43 to initiate step 216 of operating in the haptic mode HM, such haptic and/or audible feedback may indicate that the end effector 20 is aligned with the target trajectory T and that the operator may release the foot pedal to initiate step 216.

As yet another example, the method 200 may include a step of implementing haptic and/or audible feedback in response to the end effector 20 moving above the point P₃ during step 216. In instances where the controller 30 ceases operation in the haptic mode HM in response to movement of the end effector 20 above the point P₃, such haptic and/or audible feedback may indicate that the end effector 20 has moved above the point P₃ and that the robotic arm 18A has ceased operation in the haptic mode HM.

h. Displaying Virtual Representations

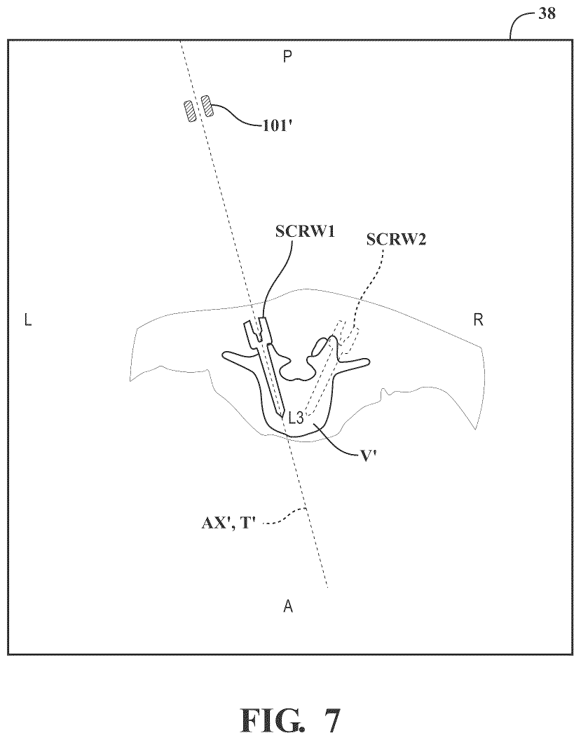

The method 200 may include a step of displaying a virtual representation of a component of the surgical system 10 or a virtual representation of a haptic object defined by the controller 30. For example, a display may be configured to display a virtual representation of the end effector 20, a virtual representation of anatomy A of the patient 12 (e.g. one or more vertebral bodies of the vertebra V), a virtual representation of the target trajectory T and/or a virtual representation of the trajectory selection zone SZ. Referring to FIG. 7, the display 38 displays a virtual representation of the guide tube 101′, a virtual representation of the axis AX′ of the guide tube 101, a virtual representation of the vertebra V′, and virtual representations of a first and second target trajectory T₁′, T₂′. Additionally, the display may be configured to display the virtual representation of the end effector 20 based on a pose of the end effector 20 and the virtual representation of anatomy A of the patient 12 and the target trajectory T based on a pose of the anatomy A. In this way, the display may continually update a position of the virtual representation of the end effector 20 based on a pose of the end effector 20, a position of the virtual representation of anatomy A, and/or a position of the target trajectory T associated with the anatomy A based on a pose of the anatomy A.

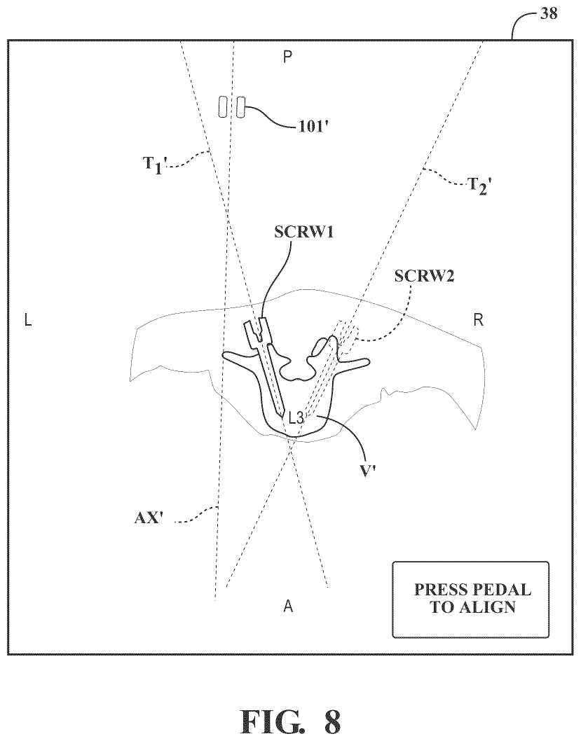

In some instances, the display may provide a virtual representation of a planned screw. For example, referring to FIGS. 7 and 8, the display 38 provides a first planned screw icon SCRW1 and a second planned screw icon SCRW2, indicating a position and orientation of a first and second screw relative to the vertebra V should the first and second screw be inserted along the first and second target trajectories T₁, T₂. In such instances, the first planned screw icon SCRW1 corresponds to the first target trajectory T₁ and the second planned screw icon SCRW2 corresponds to the second target trajectory T₂.

The display may be configured to display multiple views of the surgical system 10. For example, in FIGS. 7 and 8, the display 38 displays a cross-sectional view of the guide tube 101 and a cross-sectional view of the vertebra V. In other instances, the display may alternatively, or additionally, display any other suitable view of the surgical system 10 including, but not limited to a front, back, right, left, top, bottom, and angled views of the surgical system 10.

The step of displaying a virtual representation of a component or a haptic object may include providing an indication to an operator of the surgical system 10 via the display. The indication may be provided using any suitable icon, including but not limited to text, a bolded/highlighted icon, a color icon, and/or a blinking icon.

The display may provide an indication related to the trajectory selection zones SZ. For example, the display may provide an indication that the end effector 20 is within a trajectory selection zone SZ. In such instances, the display may provide a blinking virtual representation of the trajectory selection zone SZ. The display may also provide an indication that the end effector 20 is within a trajectory selection zone SZ in the form of an instruction. For instance, referring to FIG. 8, the display 38 provides an instruction, “PRESS PEDAL TO ALIGN”, to the operator to indicate that the end effector 20 is within a trajectory selection zone SZ and that the associated target trajectory T has been selected. The operator may then press the user-actuatable foot pedal of the footswitch 43 to confirm selection of the target trajectory T and initiate step 214 of automatically aligning the end effector 20 with the associated target trajectory T₁. As another example, the display may provide an indication that the end effector 20 is closer to the one of the trajectory selection zones SZ. For instance, the display 38 may indicate that the end effector 20 is closer to the trajectory selection zone SZ1 than the trajectory selection zone SZ2 of FIG. 5A by providing a blinking virtual representation of the trajectory selection zone SZ1.

The display may provide an indication related to the target trajectories T.

For example, the display may indicate that a target trajectory T has been selected. In such instances, the display may provide a blinking virtual representation of the selected target trajectory T. Additionally, or alternatively, the display may provide a bolded/highlighted representation of the trajectory selection zone SZ associated with the target trajectory T, the planned screw icon corresponding to the selected target trajectory T, and/or the vertebral body associated with the target trajectory T.

As another example, the display may indicate that the end effector 20 is aligned with the target trajectory T. Referring to FIG. 7, the display 38 may provide a colored virtual representation of the guide tube 101′ to indicate that the end effector 20 and the guide tube 101 are aligned with the target trajectory T. Additionally, the display 38 superimposes the axis AX of the guide tube 101 and the target trajectory T to indicate alignment of the guide tube 101 and the target trajectory T. The display may provide a bolded/highlighted representation of the target trajectory T and/or the trajectory selection zone SZ associated with the target trajectory T. Additionally, or alternatively, the display may provide a bolded/highlighted representation of the planned screw icon corresponding to the selected target trajectory T and/or the vertebral body associated with the target trajectory T, as shown in FIG. 7. The display may also provide an indication that the end effector 20 is aligned with the target trajectory T in the form of an instruction. For instance, the display 38 may provide an instruction, “RELEASE PEDAL”, to the operator to indicate that the end effector 20 is aligned with the target trajectory T and that the operator may release the user-actuatable foot pedal of the footswitch 43 and initiate step 216 of operation in the haptic mode HM.

As yet another example, the display may provide an indication that the end effector 20 is closer to one of the target trajectories T. For example, referring to FIG. 8, the first and second planned screw icons SCRW1, SCRW2 are provided to indicate that the first and second trajectories T₁, T₂ are the target trajectories T closest to the end effector 20. As shown, the first screw icon SCRW1 is bolded/highlighted to indicate that the end effector 20 is closer to the first trajectory T₁ than the second trajectory T₂. In other instances, the display may provide a blinking representation of the target trajectory T closest to the end effector 20. Additionally, or alternatively, the display may provide a bolded/highlighted representation the trajectory selection zone SZ associated with the target trajectory T closest to the end effector 20, the planned screw icon corresponding to the selected target trajectory T closest to the end effector 20, and/or the vertebral body associated with the target trajectory T closest to the end effector 20.

The display may indicate an operation mode of the robotic arm 18A. For example, the display 38 may indicate that the robotic arm 18A is operating in the free mode FM, the automatic mode AM, or the haptic mode HM. In such an instance, the display may provide an information window indicating an operating mode of the robotic arm 18A.

The step of displaying a virtual representation of a component or a haptic object may occur prior to, during, or after any step of the method 200. For example, the step of displaying a virtual representation may occur during operation of the robotic arm 18A in the free mode FM during step 210, operation of the robotic arm 18A in the automatic mode during step 214, and/or operation of the robotic arm 18A in the haptic mode HM. Additionally, the step of displaying a virtual representation may occur prior to operation of the robotic arm 18A in the free mode FM and persist throughout the method 200. Referring to FIG. 6, the display 38 may indicate that the end effector 20 is aligned with a target trajectory T while the robotic arm 18A operates in the haptic mode HM. Referring to FIG. 7, the display 38 may indicate a target trajectory T close to the end effector 20 based on the pose of the end effector 20 while the robotic arm 18A operates in the free mode FM. In this way, as an operator directs movement of the end effector 20 during operation in the free mode FM, the display 38 provides, in real-time, an indication of a target trajectory T close to the end effector 20.

### III. Additional Features

Methods 300, 400 describe additional features of the surgical system 10. Any of the methods 300, 400 may be implemented separately, or as part of the method 200 of aligning the end effector 20 to a target trajectory defined by the boundary generator 66. For example, steps of one or more of the methods 300, 400 may be incorporated as part of the method 200 by being included prior to, during, or after any step 202-216 of the method 200. Additionally, the additional features may include any suitable steps of the method 200.

a. Pull-Away Prevention

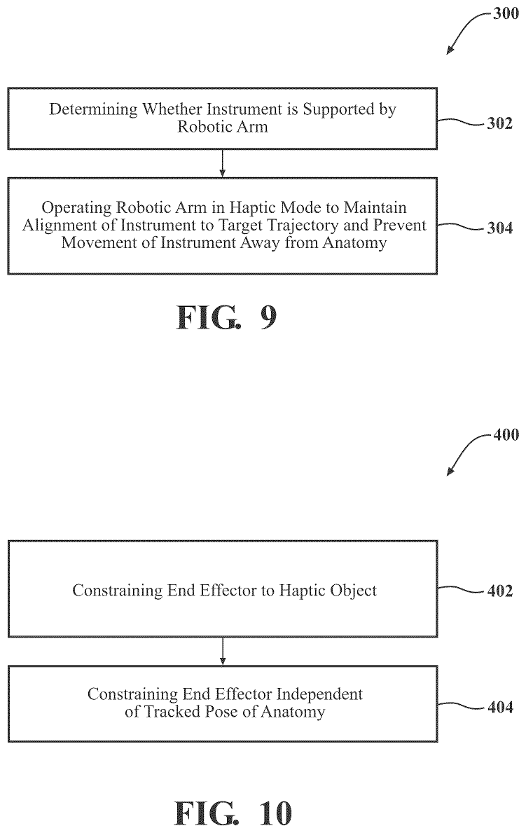

In instances where the end effector 20 includes the guide tube 101, the surgical system 10 may provide a method 300 of preventing movement of the guide tube 101 away from the anatomy A of the patient 12. As shown in FIG. 9, the method 300 may include a step 302 of determining whether a surgical instrument 110 is temporarily affixed to the guide tube 101; and a step 304 of operating the robotic arm 18A in a haptic mode HM to constrain movement of the guide tube 101 to the target trajectory T and prevent movement of the guide tube away 101 from the anatomy A of the patient 12.

The controller 30 may determine whether a surgical instrument 110 is temporarily affixed to the guide tube 101 during step 302. The controller 30 may be configured to determine whether a surgical instrument 110 is temporarily affixed to the guide tube 101 based on the navigation system 32 tracking a pose of the surgical instrument 110. For example, in instances where the tool tracker 106 is coupled to the end effector 20 via the surgical instrument 110, such as the instance of FIG. 1, the navigation system 32 may be configured to track a pose of the surgical instrument 110 by tracking the tool tracker 106. The controller 30 may be configured to determine whether a surgical instrument 110 is temporarily affixed to the guide tube 101 based on a sensing system sensing that the surgical instrument 110 is temporarily affixed to the guide tube 101. The sensing system may include any suitable components or sensor for sensing a surgical instrument 110 temporarily affixed to the guide tube 101. Additionally, the sensing system may be disposed at any suitable location of the surgical system 10. For example, the sensing system may include a hall sensor disposed within the guide tube 101, the hall sensor being configured to sense a magnetic field generated by the surgical instrument 110 when the surgical instrument 110 is temporarily affixed to the guide tube 101. As another example, the sensing system may include a force/torque sensor disposed within the robotic arm 18A, the force/torque sensor being configured to sense forces/torques applied by the surgical instrument 110 to the robotic arm 18A when the surgical instrument 110 is temporarily affixed to the guide tube 101. As another example, the controller 30 may include the sensing system and the sensing system may be configured to sense that a surgical instrument 110 is temporarily affixed to the guide tube 101 based on kinematic data of the manipulator 14.

The controller 30 may be configured to operate the robotic arm 18A in the haptic mode HM during step 304 in response to determining that a surgical instrument 110 is temporarily affixed to the guide tube 101 and in response to the guide tube 101 being aligned with the target trajectory T. The guide tube 101 may be aligned with the target trajectory T using any suitable method or steps described herein. For example, the guide tube 101 may be aligned with the target trajectory T using any of the steps of the method 200.

During step 304, the controller 30 may be configured to operate the robotic arm 18A in the haptic mode HM to constrain movement of the guide tube 101 to the target trajectory T and prevent movement of the guide tube 101 away from the anatomy A of the patient 12. In other words, movement of the guide tube 101 may be constrained during step 304 such that the guide tube 101 may move along the target trajectory T toward the anatomy A, but the guide tube 101 may not move away from the target trajectory T, and the guide tube 101 may not move away from the anatomy A of the patient 12.

The system 10 may include any suitable component for preventing movement of the guide tube 101 away from the anatomy A of the patient 12 during step 302. For example, in some instances, the robotic arm 18A may include brakes and the controller 30 may include a braking system configured to actuate the brakes. In such an instance, the controller 30 may monitor the forces/torques placed on the robotic arm 18A and/or end effector 20 by an operator to determine a commanded position of the robotic arm 18A and/or end effector 20. In an instance where the controller 30 determines that the commanded position would move the guide tube 101 away from the anatomy A, the controller 30 may control the braking system to actuate the brakes of the robotic arm 18A, preventing movement of the guide tube 101 away from the anatomy A.

In some instances, the method 300 may include the previously described step of implementing haptic and/or audible feedback to a component of the surgical system 10. Additionally, the step of implementing haptic and/or audible feedback prior to, during, or after any of the steps 302-304 of the method 300 to provide an indication to an operator. For example, the method 300 may include a step of implementing haptic and/or audible feedback in response to the controller 30 determining that a surgical instrument 110 is temporarily affixed to the guide tube 101 during step 302. As another example, the method 300 may include a step of implementing haptic and/or audible feedback in response to the controller 30 initiating operation of the haptic mode HM during step 304.

Advantageously, the method 300 prevents movement of the guide tube 101 in instances where an operator removes the surgical instrument 110 from the guide tube 101. In such instances, the surgical instrument 110 may apply frictional forces/torques to the guide tube 101 during removal of the surgical instrument 110, which may cause unintended movement of the guide tube 101 away from the anatomy A and/or away from the target trajectory T. In instances where a second surgical instrument 110 is to be temporarily affixed to the guide tube 101 after a first surgical instrument 110 is removed from the guide tube 101, unintended movement of the guide tube 101 may require realignment of the guide tube 101 to the target trajectory T. The method 300 of preventing movement of the guide tube 101 allows an operator to remove the surgical instrument 110 without causing unintended movement of the guide tube 101. In this way, the method 300 facilitates removal of a surgical instrument 110 while preventing a loss of alignment of the guide tube 101 and the target trajectory T during the removal.

The controller 30 may be configured to perform steps 302 and 304 as part of the method 200. For example, the steps 302 and 304 may be performed as an alternative to, or an addition to, step 216 of operating the robotic arm 18A in the haptic mode HM of method 200.

b. Anatomy Independent Constraint

The surgical system 10 may provide a method 400 of constraining the end effector 20 during operation of the robotic arm 18A. Generally, during the method 400, the controller 30 may be configured to constrain end effector 20, wherein such constraint is independent of a tracked pose of the anatomy A. Advantageously, such constraint provides an operator with a stable means of performing surgical procedures, as variations in tracking of the anatomy A and/or movement of the anatomy A and/or robotic arm 18A relative to one another do not affect the pose of the haptic object. In other words, the controller 30 is not updating a position of the robotic arm 18A based on dynamic tracking of the patient 12 so as to avoid jerky movements of the robot arm 18A responsive to small movements of the patient 12.

In some instances, the haptic object of method 400 may be a target trajectory T. In other instances, the haptic object may be any haptic object defined by the controller 30. For example, the haptic object may be a virtual boundary (VB), virtual mesh, virtual constraint, or the like.

As shown in FIG. 10, the method 400 may include a step 402 of constraining the end effector 20 to a haptic object associated with an anatomy A of a patient 12 and a step 404 of constraining the end effector 20 independent of the tracked pose of the anatomy A. The controller 30 may operate the robotic arm 18A in the haptic mode HM to constrain the end effector 20 during steps 402 and 404 of the method. Suitable features of the above-described haptic mode HM, such as movement along a target trajectory T, movement along a target trajectory T based on a position of one or more points, and/or pull-away prevention may be included as part of the method 400.

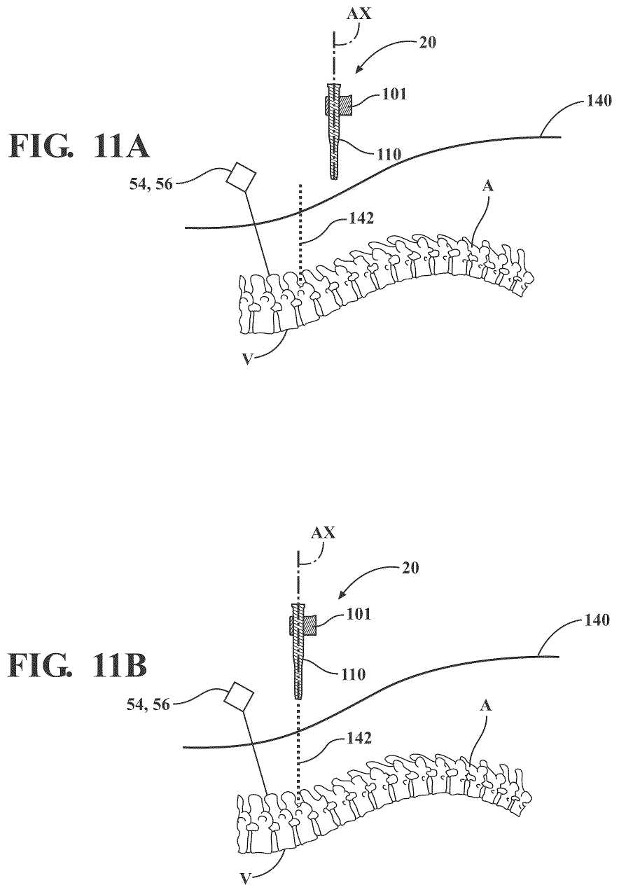

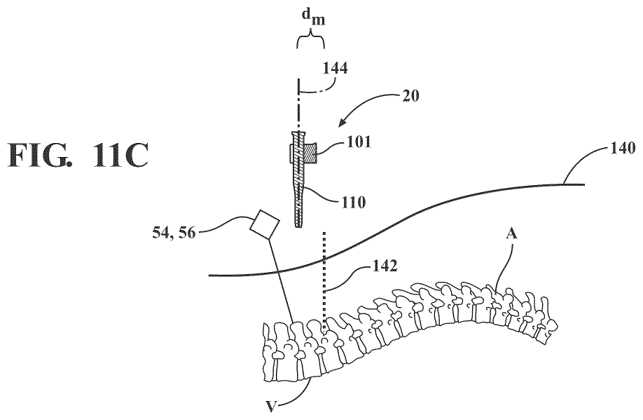

Referring to FIG. 11A-11E, the navigation system 32 tracks the patient marker 54, 56 to track a pose of the vertebra V in the localizer coordinate system LCLZ. Additionally, the robotic arm 18A supports and moves the end effector 20 in the manipulator coordinate system MNPL. The controller 30 may be configured to associate the haptic object with the anatomy A in the localizer coordinate system LCLZ. For example, referring to FIG. 11A, a haptic object 142 is associated with the vertebra V in the localizer coordinate system LCLZ. The method 400 may include any suitable steps of the method 200 to associate the haptic object 142 with the vertebra V. For example, in instances where the haptic object 142 is a target trajectory T, the method 400 may include the step 202 of tracking a pose of the end effector 20 supported by the robotic arm 18A; the step 204 of tracking a pose of an anatomy A of a patient; and the step 206 of associating a target trajectory T with the anatomy A of the patient. Additionally, the haptic object may be associated with any suitable anatomy A of the patient 12.

The end effector 20 may be moved relative to the haptic object 142 prior to being constrained to the haptic object during steps 402 and 404. In the instance of FIG. 11A-11E, the haptic object 142 is illustrated as a target trajectory. In instances where the haptic object 142 is defined as a target trajectory, prior to constraint of the end effector 20 to the haptic object 142, the end effector 20 may be aligned with the haptic object 142, as shown in FIG. 11B. In the instance of FIG. 11B, the end effector 20 includes an axis AX, and the end effector 20 is aligned with the haptic object 142 such that the axis AX is aligned with the haptic object 142.

Constraint of the end effector 20 during steps 402 and 404 may be in response to the end effector 20 being aligned with the haptic object 142. In instances where the haptic object 142 is a target trajectory, the method 400 may include a step of operating the robotic arm 18A in the free mode FM and/or automatic mode AM to align the end effector 20 with the haptic object 142. Additionally, in instances where the haptic object 142 is a target trajectory and the controller 30 defines a trajectory selection zone associated with the haptic object 142, the method 400 may include steps of the method 200 to align the end effector 20 with the haptic object 142 based on the end effector 20 being within the trajectory selection zone. For example, the method 400 may include the step 208 of defining a trajectory selection zone associated with the haptic object 142; the step 210 of operating the robotic arm 18A in the free mode FM; the step 212 of determining that the end effector 20 is within the trajectory selection zone; and the step 214 of operating the robotic arm 18A in the automatic mode AM to align the end effector 20 with the haptic object 142. Alternatively, in instances where the haptic object 142 is a virtual boundary (VB), a virtual mesh, a virtual constraint, or the like, the method 400 may include a step of operating the robotic arm 18A in the free mode FM and/or automatic mode AM to move the end effector 20 to a target site defined by the haptic object 142 prior to constraining the end effector 20 to the haptic object 142.

The controller 30 may be configured to perform step 402 of constraining the end effector 20 to the haptic object 142. In instances where the haptic object 142 is a target trajectory T, the end effector 20 may be constrained to the target trajectory T during step 402. In instances where the haptic object 142 is a virtual boundary (VB), virtual mesh, virtual constraint, or the like, movement of the end effector 20 may be constrained to an area defined by the haptic object 142. A pose of the haptic object 142 may be dependent on the tracked pose of the anatomy A. As such, during step 402, constraint of the end effector 20 may also be dependent on the tracked pose of the anatomy A. As follows, during step 402, movement of the robotic arm 18A and/or the anatomy A relative to one another may affect a pose of the haptic object 142 and constraint of the end effector 20 to the haptic object 142.

The controller 30 may be configured to perform the step 404 of constraining the end effector 20 independent of the tracked pose of the anatomy A. During step 404, movement of the robotic arm 18A and/or the anatomy A relative to one another may affect a pose of the haptic object 142, however, as constraint of the end effector 20 is independent of the tracked pose of the anatomy A, such movement would not affect constraint of the end effector 20. In other words, during step 404, movement of the end effector 20 is constrained independent of the tracked pose of the anatomy A such that an orientation of the end effector 20 does not change when the pose of the haptic object 142 changes. For example, referring to FIG. 11C, movement of the robotic arm 18A and/or the anatomy A relative to one another affects a pose of the haptic object 142. However, as shown in FIG. 11C, the controller 30 continues to constrain the end effector 20 such that an orientation of the end effector 20 does not change.

In one instance, to constrain the end effector 20 to the haptic object 142 independent of the tracked pose of the anatomy A during step 404, the controller 30 may be configured to constrain the end effector 20 to an orientation of the axis of the end effector 20 at a time of alignment of the axis of the end effector 20 with the haptic object 142. FIG. 11B illustrates an example time of alignment of the axis AX of the end effector 20 with the haptic object 142. As follows, the controller 30 may be configured to constrain the end effector 20 to an orientation of the axis AX during step 404. For example, referring to FIG. 11C, movement of the robotic arm 18A and/or the anatomy A relative to one another affects a pose of the haptic object 142. However, as shown in FIG. 11C, the controller 30 continues to constrain the end effector 20 to an orientation of the axis AX.

It is contemplated that a position of the end effector 20 may be altered while constrained by the controller 30 in step 404. As previously stated, during step 404, the controller 30 constrains the end effector such that an orientation of the end effector 20 does not change. However, a position of the end effector 20 may be altered during such constraint, provided that the orientation of the end effector 20 is not altered. For example, in an instance where the controller 30 constrains the end effector 20 to the orientation of the axis AX, the end effector 20 may move along the axis AX during step 404.

While the controller 30 constrains the end effector 20 during step 404, the controller 30 may be configured to monitor input from the navigation system 32 to determine a pose of the haptic object 142. As previously stated, movement of the robotic arm 18A and/or the anatomy A relative to one another may affect a pose of the haptic object 142. While such alterations to the pose of the haptic object 142 would not affect constraint of the end effector 20 during step 404, the controller 30 may be configured to determine the pose of the haptic object 142 during constraint.

Additionally, by monitoring the pose of the haptic object 142, the controller 30 may be configured to determine whether the robotic arm 18A and/or the anatomy A has moved relative to one another. For example, in an instance where the controller 30 constrains the end effector 20 to an orientation of the axis of the end effector 20 during step 404, the controller 30 may be configured to determine whether the robotic arm 18A and/or the anatomy A has moved relative to one another by determining a displacement between the axis AX of the end effector 20 and the haptic object 142. For instance, referring to FIG. 11C, displacement dₘ represents a displacement between the axis AX and the haptic object 142.

The controller 30 may be configured to evaluate the displacement between the axis AX of the end effector 20 and the haptic object 142 relative to a realignment threshold. For example, in some instances the realignment threshold may be a range. In such instances, the controller 30 may be configured to evaluate whether the displacement is within the range, exceeds the range, or below the range. In instances where the controller 30 determines that the displacement is within the range, the controller 30 may realign the end effector 20 with the haptic object 142 in response to determining that the displacement is within the range. The controller 30 may realign the end effector 20 using any suitable method described herein. For example, the controller 30 may operate the robotic arm 18A in the automatic mode AM, whereby the robotic arm 18A is automatically moved to align the end effector 20 with the haptic object 142. The controller 30 may also operate the robotic arm 18A in the free mode FM to allow the operator to manually move the robotic arm 18A to realign the end effector 20 with the haptic object 142. The controller 30 may then constrain the end effector 20 to the haptic object 142 in accordance with step 402 or constrain the end effector 20 independent of the tracked pose of the anatomy A in accordance with step 404. In instances where the controller 30 determines that the displacement exceeds the range, the controller 30 may be configured to cease constraint of the end effector 20 in response to determining that the displacement exceeds the range. As an example, the controller 30 may cease constraint of the end effector 20 and operate the robotic arm 18A in the free mode FM, whereby the robotic arm 18A is freely moveable. In instances where the controller 30 determines that the displacement is below the range, the controller 30 may be configured to continue constraining the end effector 20 independent of the tracked anatomy A in accordance with step 404.

It is contemplated that the realignment threshold may be selected based on predetermined distances to provide for suitable operation of the controller 30. For example, in instances where the realignment threshold is a range, the value corresponding to the minimum of the range may be selected to prevent the controller 30 from realigning the end effector 20 based on noise received from the navigation system 32. Additionally, the value corresponding to the maximum of the range may be selected to prevent the robotic arm 18A from moving a substantial distance during realignment of the end effector 20.

Additionally, it is contemplated that, the controller 30 may be configured to evaluate the displacement the axis AX of the end effector 20 and the haptic object 142 using any suitable method and/or means. For example, the realignment threshold may be a value, instead of a range, and the controller 30 may be configured to evaluate the displacement relative to the value. Additionally, the controller 30 may be configured to evaluate the displacement between the axis AX of the end effector 20 and the haptic object 142 relative to more than one threshold range/value. For example, the controller 30 may be configured to cease constraint of the end effector 20 in response to determining that the displacement exceeds a movement threshold value, and the controller 30 may be configured to realign the end effector 20 with the haptic object 142 in response to determining that the displacement exceeds a realignment threshold value.

In some instances, the method 400 include a step of detecting an input from the one or more input devices 40, 42, 43. In such instances, any one or more of the steps 402, 404 of the method 400 may occur in response to the step of detecting an input from the one or more input devices 40, 42, 43. For example, the step 402 of constraining the end effector 20 to the haptic object 142, may occur in response to detecting an input from the one or more input devices 40, 42, 43. As another example, the step 404 of constraining the end effector 20 independent of the tracked pose of the anatomy A, may occur in response to detecting an input from the one or more input devices 40, 42, 43. As another example, the controller 30 may be configured to operate the robotic arm in the automatic mode AM to realign the end effector 20 with the haptic object 142 in response to determining that a displacement between the axis AX of end effector 20 and the haptic object 142 is within a realignment threshold and in response to detecting an input from the one or more input devices 40, 42, 43. As another example, the controller 30 may be configured to constrain the end effector 20 after realigning the end effector 20 with the haptic object 142 in response to detecting an input from the one or more input devices 40, 42, 43. In an example instance, an operator may press the user-actuatable foot pedal of the footswitch 43 to initiate step 402, and the operator may press the user-actuatable foot pedal of the footswitch 43 once more to initiate step 404. In another example instance, an operator may hold the user-actuatable foot pedal of the footswitch 43 to initiate step 402, and the operator may release the user-actuatable foot pedal of the footswitch 43 to initiate step 404. In another example instance, in response to the controller 30 determining that movement of the anatomy and/or robotic arm relative to one another is within a realignment threshold, the operator may hold the user-actuatable foot pedal of the footswitch 43 to realign the end effector 20 with the haptic object 142, and the operator may release the user-actuatable foot pedal of the footswitch 43 to constrain the end effector 20 to the haptic object 142.

The method 400 may include a step of displaying a virtual representation of a component of the surgical system 10 or a virtual representation of a haptic object 142 defined by the controller 30. For example, a display may be configured to display a virtual representation of the end effector 20, a virtual representation of anatomy A of the patient 12 (e.g. one or more vertebral bodies of the vertebra V), a virtual representation of the haptic object 142. Additionally, the display may provide an indication related to the haptic object 142. For example, the display may provide an indication that the end effector 20 is constrained to the haptic object 142 in accordance with step 402. As another example, the display may provide an indication that the end effector 20 is constrained independent of the tracked anatomy A in accordance with step 404. As another example, the display may provide a virtual representation of the displacement between the axis AX of the end effector 20 and the haptic object 142. The display may also provide an instruction, “PATIENT HAS MOVED”, to the operator to indicate that the displacement between the axis AX of the end effector 20 and the haptic object 142 is within the realignment threshold and that the end effector 20 may be realigned with the haptic object 142. The display may also provide an instruction, “PRESS PEDAL TO ALIGN”, to the operator to indicate that the displacement between the axis AX of the end effector 20 and the haptic object 142 is within the realignment threshold and that the end effector 20 may be realigned with the haptic object 142. The display may also provide an instruction, “REMOVE INSTRUMENT FROM PATIENT”, prior to realignment of end effector 20 with the haptic object 142 to warn the operator to remove the surgical instrument before the end effector 20 is moved during realignment. The display may also provide a warning, “MOVEMENT THRESHOLD EXCEEDED”, prior to ceasing constraint of end effector 20 and in response to determining that the displacement between the axis AX of the end effector 20 and the haptic object 142 has exceeded the realignment threshold.

The controller 30 may be configured to perform steps 402 and 404 as part of the method 200. For example, the steps 402 and 404 may be performed as an alternative to, or an addition to, step 216 of operating the robotic arm 18A in the haptic mode HM of method 200.

The techniques described above can be utilized for other surgical procedures besides spinal procedures and can be utilized with end effector(s) or surgical instrument(s) other than those shown and described. For example, selection zones can be utilized in a total knee replacement procedure whereby virtual cutting planes are associated with various selection zones. In a shoulder replacement procedure, virtual screw axes can be associated with various selection zones.

Although specific features of various embodiments of the disclosure may be shown in some drawings and not in others, this is for convenience only. In accordance with the principles of the disclosure, any feature of a drawing or other embodiment may be referenced and/or claimed in combination with any feature of any other drawing or embodiment.

This written description uses examples to describe embodiments of the disclosure and also to enable any person skilled in the art to practice the embodiments, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. A surgical system comprising:
a robotic arm comprising a plurality of links and joints and being configured to support an end effector;
a navigation system configured to track a pose of an anatomy of a patient; and
one or more controllers configured to:
associate a target trajectory with the anatomy of the patient;
define a trajectory selection zone associated with the target trajectory;
operate the robotic arm in a free mode, whereby the robotic arm is freely moveable;
responsive to the end effector being within the trajectory selection zone in the free mode, automatically select the target trajectory associated with the trajectory selection zone; and
operate the robotic arm in an automatic mode, whereby the robotic arm is automatically moved to align the end effector with the target trajectory.

2. The surgical system of claim 1, wherein the one or more controllers are further configured to, responsive to the end effector being aligned with the target trajectory, operate the robotic arm in a haptic mode, whereby movement of the end effector is constrained to the target trajectory.

3. The surgical system of claim 1, wherein the one or more controllers are further configured to:
define a first point and a second point along the target trajectory, wherein the first point is located at a first position along the target trajectory, wherein the second point is located at a second position along the target trajectory, and wherein the first position is further from the anatomy than the second position;
responsive to end effector being aligned with the target trajectory, operate the robotic arm in a haptic mode, whereby movement of the end effector is constrained to the target trajectory above the second point; and
responsive to movement of the end effector along the target trajectory above the first point during operation of the robotic arm in the haptic mode, cease operation of the robotic arm in the haptic mode.

4. The surgical system of claim 1, further comprising a display and an input device, and wherein the one or more controllers are further configured to:
in response to automatic selection of the target trajectory, notify a user of the automatically selected target trajectory via the display;
in response to notifying the user, detect an input from the input device; and
in response to detection of the input, operate the robotic arm in the automatic mode to automatically align the end effector with the automatically selected target trajectory.

5. The surgical system of claim 1, further comprising an input device, and wherein the one or more controllers are further configured to:
detect an input from the input device; and
responsive to detection of the input and responsive to the end effector being aligned with the target trajectory, operate the robotic arm in a haptic mode, whereby movement of the end effector is constrained to the target trajectory.

6. The surgical system of claim 1, wherein the one or more controllers are further configured to implement haptic feedback to indicate that the target trajectory is selected.

7. The surgical system of claim 1, wherein the one or more controllers are further configured to:
associate a prevention zone with the target trajectory, and
responsive to the end effector being within the trajectory selection zone and the prevention zone in the free mode, automatically select the target trajectory associated with the trajectory selection zone.

8. The surgical system of claim 1, wherein the target trajectory is further defined as a first target trajectory, and wherein the one or more controllers are further configured to associate a second target trajectory with the anatomy of the patient.

9. The surgical system of claim 8, wherein the trajectory selection zone is further defined as a first trajectory selection zone, and wherein the one or more controllers are further configured to:
associate a second trajectory selection zone with the second target trajectory; and
responsive to the end effector being within the second trajectory selection zone in the free mode, automatically select the second target trajectory associated with the second trajectory selection zone.

10. The surgical system of claim 9, wherein a size of the first trajectory selection zone is equivalent to a size of the second trajectory selection zone.

11. The surgical system of claim 9, further comprising a display, wherein the one or more controllers are further configured to determine whether the end effector is closer to the first target trajectory or the second target trajectory and wherein the display is configured to indicate whether the end effector is closer to the first target trajectory or the second target trajectory.

12. The surgical system of claim 9, further comprising a display configured to indicate whether the end effector is closer to the first trajectory selection zone or the second trajectory selection zone.

13. The surgical system of claim 1, further comprising a display configured to present the selected target trajectory, wherein:
the display is configured to provide at least one of a virtual representation of the selected target trajectory, a virtual representation of a planned screw corresponding to the selected target trajectory, a virtual representation of the anatomy associated with the selected target trajectory, and a virtual representation of the trajectory selection zone associated with the selected target trajectory; and
the display is configured to highlight the at least one of the virtual representation of the selected target trajectory, the virtual representation of the planned screw corresponding to the selected target trajectory, the virtual representation of the anatomy associated with the selected target trajectory, and the virtual representation of the trajectory selection zone associated with the selected target trajectory.

14. The surgical system of claim 1, further comprising a display configured to indicate that the end effector is aligned with the target trajectory. 15 The surgical system of claim 1, wherein the end effector includes a guide tube configured to support an instrument temporarily affixed to the guide tube.

16. The surgical system of claim 15, wherein the one or more controllers are configured to:
determine whether an instrument is temporarily affixed to the guide tube; and
responsive to determining that an instrument is temporarily affixed to the guide tube and responsive to the guide tube being aligned with the target trajectory, operate the robotic arm in a haptic mode to constrain movement of the guide tube to the target trajectory and prevent movement of the guide tube away from the anatomy of the patient.

17. The surgical system of claim 1, further comprising an input device, wherein a pose of the target trajectory is dependent on the tracked pose of the anatomy, and wherein the one or more controllers are further configured to:
responsive to detecting a first input from the input device and responsive to the end effector being aligned with the target trajectory, constrain the end effector to the target trajectory; and
responsive to detecting a second input from the input device and responsive to the end effector being aligned with the target trajectory, constrain the end effector independent of the tracked pose of the anatomy.

18. The surgical system of claim 17, wherein the one or more controllers are configured to constrain the end effector independent of the tracked pose of the anatomy such that an orientation of the end effector does not change when the pose of the target trajectory changes.

19. The surgical system of claim 17, wherein the end effector extends along an axis, and wherein, to constrain movement of the end effector independent of the tracked pose of the anatomy, the one or more controllers are configured to constrain the end effector to an orientation of the axis of the end effector at a time of alignment of the axis of the end effector with the target trajectory.

20. The surgical system of claim 17, wherein the one or more controllers are configured to monitor input from the navigation system to determine the pose of the target trajectory.
